(19) United States
(12) Patent Application Publication
(10) Pub. No.: US 2025/0354913 A1
(43) Pub. Date: Nov. 20, 2025

(54) MULTIMODAL AND MODULAR APPARATUS FOR OPTICAL MEASUREMENTS OF A MATERIAL SAMPLE

(71) Applicant: RHÉOLUTION INC., Montréal (CA)

(72) Inventors: Anis Redha Hadj Henni, Montréal (CA); Cedric Schmitt, Montréal (CA); Elie Haddad, Montréal (CA); Jean-Philippe Tremblay, Montréal (CA)

(73) Assignee: RHÉOLUTION INC., Montréal, QC (CA)

(21) Appl. No.: 19/279,867

(22) Filed: Jul. 24, 2025

Related U.S. Application Data
(63) Continuation of application No. PCT/CA2024/050113, filed on Jan. 30, 2024.
(60) Provisional application No. 63/482,503, filed on Jan. 31, 2023.

(51) Int. Cl. G01N 21/03; G01N 21/01

(52) U.S. Cl. CPC G01N 21/0303; G01N 2021/0112; G01N 2201/0245

(57) ABSTRACT

A multimodal and modular optical apparatus is provided for acquiring optical data and generating at least one parameter for characterization of a material sample. A principal system includes a main body defining a sample receiving space and at least one cartridge-receiving space, at least one cartridge being sized and shaped for reversible insertion in one cartridge-receiving space, a cartridge connector configured to communicate the optical data, and a control and processing unit being in data communication with the at least one cartridge via the cartridge connector to receive the optical data. The apparatus can further include at least one module being operatively connected to the main body and being in data communication with the control and processing unit of the principal system. A method to characterize a material sample based on optical data acquired by an apparatus having at least one optical measurement modality is further provided.

This patent application is a continuation of International Application No. PCT/CA2024/050113, filed on Jan. 30, 2024, which claims the benefit of priority to U.S. Provisional Patent Application No. 63/482,503, filed Jan. 31, 2023, the entire teachings and disclosures of both applications are incorporated herein by reference thereto.

### FIELD OF THE INVENTION

The present invention generally relates to apparatuses and methods for measuring optical properties, and more particularly to a multimodal system and related modular assembly performing optimal measurements and generating at least one property for characterizing a material sample.

### BACKGROUND

Modern research and development and scientific research are more and more interdisciplinary and complex. In order to innovate, solve complex scientific problems and design effective technologies and products, scientists need to use versatile, modular, scalable and interconnected laboratory apparatuses that fit their changing needs and evolving projects. From an economic perspective, modern labs and organizations also need to optimize their investments in new equipment and instrumentation that are specifically designed to be scalable, modular and data-driven.

One of the challenges scientists and organizations are facing relates to the fact that the vast majority of commercially available laboratory testing and analysis apparatuses are not modular, are difficult to effectively scale into complementary systems, are not interconnected and are not designed to collect and organize data in a comprehensive and integrated manner.

There is thus a need for a technology that overcomes at least some of the drawbacks of what is known in the field, such as the above-mentioned drawbacks.

### SUMMARY

Implementations of an optical apparatus respond to the above need by providing a multimodal and modular system including interchangeable optical cartridges tailored to each acquire data from a material sample for a given optical measurement modality, and a control and processing unit in data communication with the optical cartridges to generate a given parameter from the optical measurement data, thereby characterizing the material sample. The principal system can serve as a base to connect at least one additional module that is configured to provide additional functional or operational modalities to the apparatus. The multimodal and modular apparatus can be integrated in an assembly including connected apparatuses to form a network.

In one aspect, there is provided a multimodal and modular optical apparatus for acquiring optical data from a material sample and generating at least one parameter for characterization of the material sample from the optical data. The apparatus comprises a principal system being configured to operate multiple optical measurement modalities. The principal system comprises a sample receiving space and at least one cartridge-receiving space, wherein the sample receiving space is sized and shaped for reversible insertion of the material sample. The sample receiving space is in optical communication with the at least one cartridge-receiving space. The principal system further comprises a cartridge assembly comprising at least one cartridge being sized and shaped for reversible insertion in the at least one cartridge-receiving space. The cartridge assembly comprises an emitter configured to emit electromagnetic radiations propagating to the material sample, and a receiver configured to receive and measure an electromagnetic response from the material sample in response to the stimulation provided by the electromagnetic radiations from the emitter. The measured electromagnetic response thus forms the optical data that conveys information regarding the material sample, and more particularly that is processable to extract at least one parameter characterizing a physical, chemical and/or biological property of the material sample.

More particularly, there is provided a multimodal and modular optical apparatus for acquiring optical data from a material sample and generating at least one parameter for characterization of the material sample from the optical data. The apparatus comprises a main body defining a sample receiving space and at least one cartridge-receiving space, wherein the sample receiving space is sized and shaped for reversible insertion of the material sample. The apparatus further comprises at least one cartridge being sized and shaped for reversible insertion in the at least one cartridge-receiving space. The at least one cartridge comprises an emitter configured to emit electromagnetic radiations propagating to the material sample, and/or a receiver configured to receive and measure electromagnetic radiations from the material sample in response to the emitter, thereby generating the optical data. The at least one cartridge further comprises a cartridge connector configured to communicate the optical data. The apparatus can further comprise a control and processing unit being in data communication with the at least one cartridge via the cartridge connector to receive the optical data, and further generating the at least one parameter for characterization of the material sample from the optical data.

The at least one cartridge and the main body can be said to be part of a principal system. The control and processing unit can be further part of the principal system of the apparatus, or alternatively the control and processing unit of the apparatus can be provided separately from the principal system and connectable to the principal system to acquire the optical data.

In some implementations, the apparatus further comprises a sample container being sized and shaped to contain the material sample, wherein at least a portion of the sample container conducts/propagates the electromagnetic radiations.

In some implementations, the at least one cartridge can comprise multiple cartridges being movable from one cartridge receiving space to another cartridge receiving space to propagate the electromagnetic radiations according to adjusted angles of emission/reflection.

In some implementations, the sample receiving space can be central to the main body and the at least one cartridge receiving space comprises multiple cartridge receiving spaces being positioned peripherally around the sample receiving space.

In some implementations, the sample receiving space can be an elongated channel and the principal system further comprises a conveyor at least partially encased in the sample receiving space, wherein the at least one cartridge receiving space comprises multiple cartridge receiving spaces being positioned on opposed longitudinal sides on the conveyor.

In some implementations, the sample receiving space can be an elongated channel and the principal system further comprises a positioning platform at least partially encased in the sample receiving space, wherein the at least one cartridge receiving space comprises multiple cartridge receiving spaces being positioned above or below the positioning platform.

In some implementations, the principal system can further comprise an analysis chamber having an aperture to receive the material sample and being insertable in the sample receiving space. Optionally, analysis chamber can comprise an actuator to expose the material sample to external stimuli; and/or a sensor to measure a response of the material sample to the external stimuli. Further optionally, the analysis chamber can further comprise a secondary control and processing unit, and a secondary connector to ensure at least one of data communication with and power supply to the control and processing unit via the secondary connector.

In some implementations, the at least one cartridge can comprise an optical window to let a light beam going out of the cartridge or to let a light beam enter in the cartridge. Optionally, the at least one cartridge can comprise an additional sensor to measure physico-chemical data from the material sample.

In some implementations, the at least one cartridge can include an emitter cartridge, a spot emitter cartridge, a linear emitter cartridge, an emitter-receiver cartridge, a receiver cartridge, a spot receiver cartridge, a linear receiver cartridge, a light stimulation cartridge, a Brownian motion cartridge, a Raman spectroscopy cartridge, a contactless temperature measurement cartridge, an imaging cartridge, or any combinations thereof.

In some implementations, the cartridge connector can ensure electric power supply to the cartridge in addition to data communication.

In some implementations, the apparatus can further comprise at least one module being operatively connected to the main body and being in data communication with the control and processing unit of the principal system. The at least one module comprises a module actuator to perform at least one automatic operation; and/or a module sensor to perform measurement of additional physico-chemical data. For example, the at least one automatic operation can comprise handling, displaying, sorting, scanning, regulating, controlling, acquiring data, storing data or any combinations thereof. For example, the physico-chemical data can comprise temperature, light, humidity, gas, image or any combinations thereof. For example, the at least one parameter can be at least one optical parameter comprising turbidity, nephelometric turbidity, optical density, absorbance, transmittance, fluorescence intensity, absorption spectra, or any combinations thereof.

In some implementations, the module can include mechanical components for hooking and/or alignment of the module with the principal system. Optionally, the module can include a secondary control and processing unit being connectable to the principal system via a universal connector for ensuring the data communication and the power supply.

In some implementations, the at least one module can include a thermal module, a battery module, a display module, an automatic platform module, a multi-identification module, a carousel dispensing module, an imaging module, a liquid circulation module, a gas injection module, a drop analysis module, a sensor module, a light stimulation module, or any combinations thereof.

In some implementations, the principal system can be configured as an absorbance meter, a transmittance meter, a colorimeter, a turbidimeter, a nephelometer, a spectrophotometer, a backscatter meter, a fluorometer, an optical plate reader, an on-line optical apparatus, or any combinations thereof.

In some implementations, the control and processing unit can be connected to an intranet or internet to form a network. Optionally, the network can further comprise a local or cloud database for storing the optical data and generated parameters.

In another aspect, there is provided an assembly comprising multiple multimodal and modular optical apparatuses as defined herein, each apparatus being configured for acquiring optical data from at least one material sample and generating at least one parameter for characterization of the at least one material sample; wherein the multiple apparatuses are in data communication with one another via their respective control and processing units.

In some implementations, the assembly can further include a database that aggregates data and metadata coming from the multiple apparatuses.

In some implementations, the assembly can further include a user device including a desktop computer, a laptop computer, a tablet, a cellphone or any combinations thereof, for communicating in real time with the multiple apparatuses through an intranet or internet.

In some implementations, the assembly can further include a robot or any other automatic platform.

In some implementations, each one of the multiple apparatuses can be configured based on different optical measurement modalities or other sensor modalities.

In another aspect, there is provided a method to characterize a material sample based on optical data acquired by an apparatus having at least one optical measurement modality. The method includes providing a material sample in a sample receiving space of the apparatus or assembly of apparatuses as defined herein; selecting the at least one optical measurement modality comprising inserting at least one cartridge in at least one cartridge receiving space of the principal system; emitting and/or receiving the electromagnetic radiations via the at least one cartridge to generate the optical data from the material sample according to the selected optical measurement modality; acquiring the optical data generated by the at least one cartridge in the control and processing unit of the principal system; and generating the at least one parameter characterizing the material sample from the optical data in the control and processing unit.

In some implementations, the method can further comprise selecting another optical measurement modality to generate another parameter characterizing the material sample by performing at least one of:
releasing the at least one cartridge from the corresponding cartridge receiving space and inserting the at least one cartridge in another cartridge receiving space of the principal system;
inserting another cartridge in another cartridge receiving space of the principal system; and
releasing the at least one cartridge from the corresponding cartridge receiving space and inserting another cartridge in said cartridge receiving space of the principal system.

In some implementations, the method can further include providing another measurement modality to generate another parameter characterizing the material sample by connecting an additional module to the principal system of the apparatus.

In another aspect, there is provided a process comprising monitoring at least one parameter characterizing a physical, chemical and/or biological property of a liquid material, wherein the monitoring includes measuring optical data from the liquid material using the multimodal and modular optical apparatus as defined herein, with the optical data being correlated to the at least one parameter. For example, the multimodal and modular optical apparatus is as defined in herein including the immersion test principal system implementation.

For example, the at least one parameter can be coagulation and the liquid material can be milk. In another example, the at least one parameter can be microbiological fermentation or enzymatic coagulation, and the liquid material can be animal milk or an alcoholic beverage during fermentation thereof. In another example, the at least one parameter can be cell proliferation and the liquid material can include at least one of cell cultures, microbes, or yeast.

In some implementations, the process further comprises communicating the monitored parameter to a control system and actuating at least one corrective action when the monitored parameter is off-specification.

While the invention will be described in conjunction with example embodiments, it will be understood that it is not intended to limit the scope of the invention to such embodiments. On the contrary, it is intended to cover all alternatives, modifications and equivalents as may be included as defined by the present description. The objects, advantages and other features of the present invention will become more apparent and be better understood upon reading of the following non-restrictive description of the invention, given with reference to the accompanying drawings.

### BRIEF DESCRIPTION OF THE DRAWINGS

Implementations of the proposed multimodal and modular optical apparatus are represented in and will be further understood in connection with the following figures.

FIG. 1 is a top perspective front view of a principal system of the multimodal and modular optical apparatus including a housing and a cover according to an embodiment of the invention.

FIG. 2 is a top perspective front view of the principal system of FIG. 1 showing the cover including an upper lid in an open position.

FIG. 3 is a top perspective rear view of the principal system of FIG. 1 showing connectors to a local network, a local storage database and a local power source.

FIG. 4 is a top perspective front and partly exploded view of the principal system of FIG. 1 showing the cover in a removed position with a plurality of optical cartridges being apparent.

FIG. 5 is a top perspective view of a bottom surface of the cover of the principal system of FIG. 1 showing notches for alignment mechanisms and hooking mechanisms for the sake of facilitating the connection of the cover to the housing.

FIG. 6 is a bottom perspective front view of the principal system of FIG. 1 showing an access panel of the housing in a removed position to reveal a lower connector.

FIG. 7A is a top semitransparent perspective front view of the principal system of FIG. 1 showing a main body within the housing and multiple cartridges inserted within the main body.

FIG. 7B is a top exploded perspective front view of the principal system shown in FIG. 7A showing cartridges and an analysis chamber in an exploded way.

FIG. 8 is a cross-sectional view along a vertical plane of the main body shown in FIG. 7A showing the analysis chamber and multiple cartridges inserted within spaces of the main body.

FIG. 9A is a top perspective front view of a spot emitter cartridge that is insertable in the principal system of the multimodal and modular optical apparatus including a housing and a cover according to an embodiment of the invention.

FIG. 9B is a top perspective rear view of the cartridge of FIG. 9A.

FIG. 10A is a top perspective front view of an emitter-receiver cartridge that is insertable in the principal system of the multimodal and modular optical apparatus including a housing and a cover according to an embodiment of the invention.

FIG. 10B is a top perspective rear view of the cartridge of FIG. 10A.

FIG. 11A is a top perspective front view of another emitter-receiver cartridge that is insertable in the principal system of the multimodal and modular optical apparatus including a housing and a cover according to an embodiment of the invention.

FIG. 11B is a top perspective rear view of the cartridge of FIG. 11A.

FIG. 12A is a top perspective front view of a linear emitter cartridge that is insertable in the principal system of the multimodal and modular optical apparatus including a housing and a cover according to an embodiment of the invention.

FIG. 12B is a top perspective rear view of the cartridge of FIG. 12A.

FIG. 13A is a top perspective front view of a spot receiver cartridge that is insertable in the principal system of the multimodal and modular optical apparatus including a housing and a cover according to an embodiment of the invention.

FIG. 13B is a top perspective rear view of the cartridge of FIG. 13A.

FIG. 14A is a top perspective front view of a linear receiver cartridge of the principal system of the multimodal and modular optical apparatus.

FIG. 14B is a top perspective rear view of the cartridge of FIG. 13A.

FIG. 15 is a top perspective front view of the multimodal and modular optical apparatus including a principal system and an additional bottom module that is connected to the principal system according to an embodiment of the invention.

FIG. 16A is a top perspective front view of the additional bottom module being a thermal module according to an embodiment of the invention.

FIG. 16B is a bottom perspective rear view of the thermal bottom module of FIG. 16A.

FIG. 17 top perspective front view of the additional bottom module being a battery module according to an embodiment of the invention.

FIG. 18A is a top perspective front view of an assembly including the additional bottom module being a display module and a connected principal system according to an embodiment of the invention.

FIG. 18B is a top perspective front view of the display module of FIG. 18A.

FIG. 19A is a top perspective side view of an assembly including the additional bottom module being an automatic platform module, an additional top module being multi-identification module, and connected principal system according to an embodiment of the invention.

FIG. 19B is a top perspective side view of the automatic platform module of FIG. 19A.

FIG. 20 is a top perspective side view of an assembly including multiple principal systems, multiple top multi-identification modules being connected to each principal system and a single bottom module being an automatic platform module connected to the multiple principal systems according to an embodiment of the invention.

FIG. 21 is a top perspective front view of an assembly including a principal system and an additional top module being a multi-identification module connected to the principal system according to an embodiment of the invention.

FIG. 22A is a top perspective front view of a bottom surface of the multi-identification module of FIG. 21.

FIG. 22B is a top perspective front view of a top surface of the multi-identification module of FIG. 21 comprising a reader in an open position.

FIG. 23 is a top perspective front view of a connectable module being a carousel dispensing module according to an embodiment of the invention.

FIGS. 24A to 24C are top perspective front views of an assembly including a principal system and a carousel dispensing module as shown in FIG. 23 being connected to the principal system for sequential operation of the multimodal and modular optical apparatus.

FIG. 25A is a top perspective view of an analysis chamber that is insertable in a main body of a principal system as shown in FIG. 7B according to an embodiment of the multimodal and modular optical apparatus.

FIG. 25B is a top perspective sectional view of the analysis chamber of FIG. 25A.

FIG. 26A is a top perspective front view of a sample container being a cuvette and a sample container adapter being a cuvette sample container adapter that is insertable in an analysis chamber as shown in FIG. 25A according to an embodiment of the invention.

FIG. 26B is a top perspective sectional view of the cuvette sample container adapter of FIG. 26A.

FIG. 26C is a top perspective bottom view of the cuvette sample container adapter of FIG. 26A.

FIG. 27A is a top perspective front view of a sample container being a vial and another sample container adapter being a vial sample container adapter according to an embodiment of the invention.

FIG. 27B is a top perspective front exploded view of an assembly including the vial sample container adapter of FIG. 27A, a vial sample container and an analysis chamber.

FIG. 27C is a top perspective bottom view of the vial sample container adapter of FIG. 27A.

FIG. 28A is a top perspective front view of a sample container being a PCR-tube and another sample container adapter being a PCR-tube sample container adapter according to an embodiment of the invention.

FIG. 28B is a top perspective bottom view of the PCR-tube sample container adapter of FIG. 28A.

FIG. 29A is a top perspective front view of another analysis chamber being a thermo-optical analysis chamber and including a connector according to an embodiment of the invention.

FIG. 29B is a top perspective bottom view of the thermo-optical analysis chamber of FIG. 29A showing an air flow inlet/outlet.

FIG. 30A is a top perspective front view of another analysis chamber being a rotating platform analysis chamber and including a connector according to an embodiment of the invention.

FIG. 30B is a top perspective sectional view of the rotating platform analysis chamber of FIG. 30A.

FIG. 31A is a top perspective view of a top surface of a carousel adapter according to an embodiment of the invention.

FIG. 31B is a top perspective view of a bottom surface of the carousel adapter of FIG. 31A.

FIG. 32 is a top perspective front view of an assembly including multiple multimodal and modular optical measurement apparatuses and a display device being connectable in a network for acquiring various optical and/or thermal data of at least one material sample, generating at least one parameter characterizing the material sample and displaying such parameter to a user according to an embodiment of the invention.

FIG. 33A is a top perspective front view of another principal system including an automatic conveyor handling platform according to an embodiment of the invention.

FIG. 33B is a top perspective front view of the principal system of FIG. 33A including a cover in an open position showing cartridges being inserted therein.

FIG. 34A is a top perspective front view of another principal system including an automatic scanning platform according to an embodiment of the invention.

FIG. 34B is a top perspective front view of the principal system of FIG. 34A including a cover in an open position showing cartridges being inserted therein.

FIG. 35 is a block diagram illustrating a multisite system architecture for operation of a plurality of remote multimodal and modular optical apparatuses.

FIG. 36 is a block diagram illustrating a single site system architecture using an internal communication network for operation of a plurality of local multimodal and modular optical apparatuses.

FIG. 37 is a block diagram illustrating a single site system architecture using an external communication network for operation of a plurality of local multimodal and modular optical apparatuses.

FIG. 38 is a block diagram illustrating operation of a multimodal and modular optical apparatus for measuring multi-modal optical and/or thermal data from a material sample in accordance with certain embodiments of the invention.

FIG. 39 is a block diagram illustrating operation of a module being connected to a principal system of a multimodal and modular optical apparatus in accordance with certain embodiments of the invention.

FIG. 40 is a block diagram of an emitter cartridge connected to the principal system of the multimodal and modular optical apparatus in accordance with certain embodiments of the invention.

FIG. 41 is a block diagram illustrating operation of an emitter-receiver cartridge connected to a principal system of a multimodal and modular optical apparatus in accordance with certain embodiments of the invention.

FIG. 42 is a block diagram illustrating operation of an analysis chamber of a principal system of a multimodal and modular optical apparatus in accordance with certain embodiments of the invention.

FIG. 43 is a block diagram illustrating operation of a receiver cartridge connected to a principal system of a multimodal and modular optical apparatus in accordance with certain embodiments of the invention.

FIG. 44 is a block diagram illustrating steps of a protocol for performing optical and other sensor measurements of a single material sample using a multimodal and modular optical apparatus in accordance with certain embodiments of the invention.

FIG. 45 is a block diagram illustrating steps of a protocol for performing optical measurement of a single material sample using a multimodal and modular optical apparatus in accordance with certain embodiments of the invention.

FIG. 46 is a block diagram illustrating steps of a protocol for performing other sensor measurements (including pH, humidity, temperature and other information) of a single material sample using a multimodal and modular optical apparatus in accordance with certain embodiments of the invention.

FIG. 47 is a block diagram illustrating steps of a protocol for performing optical and other sensor measurements as function of temporal, stimuli and additive parameter configurations using a multimodal and modular optical apparatus in accordance with certain embodiments of the invention.

FIG. 48 is a graph of a normalized light signal as a function of time of a coagulating milk sample measured at different angles with respect to a light path produced by a multimodal and modular optical apparatus in accordance with certain embodiments of the invention.

FIG. 49 is a graph of a relative intensity as a function of time of agarose gel formation samples measured by a multimodal and modular optical apparatus in accordance with certain embodiments of the invention.

FIG. 50A is a top perspective front view of another implementation of the principal system showing a sealed housing and main body for in situ measurement in a liquid material via immersion of the system.

FIG. 50B is a partial exploded view of FIG. 50A showing the housing being unsealed from the main body and revealing two cartridge-receiving spaces according to an implementation of the system.

FIG. 51 is a partial exploded top perspective view of the principal system including a sealed housing and main body for in situ measurement in a liquid material via immersion of the system, the housing being shown unsealed from the main body and revealing three cartridge-receiving spaces according to another implementation of the system.

FIG. 52 is a side and semitransparent perspective view of a vat containing a liquid material and showing the system of FIGS. 50A and 50B being immersed in the liquid material.

### DETAILED DESCRIPTION

The multimodal and modular optical apparatus is intended to characterize different properties of a material using electromagnetic radiations. The multimodal and modular optical apparatus includes a principal system comprising means for acquiring optical data from a material sample and means to generate at least one parameter that is characteristic of a physical, chemical or biological property of the material, based on the acquired optical data. In some implementations, the multimodal and modular apparatus can further include at least one module that is connected to the principal system to confer additional operational modalities and/or measurement modalities to the apparatus. Optionally, the principal system can include further means to acquire additional data (non-optical) from the material sample including physical data (such as temperature of the material sample) and/or chemical data (such as pH of the material sample).

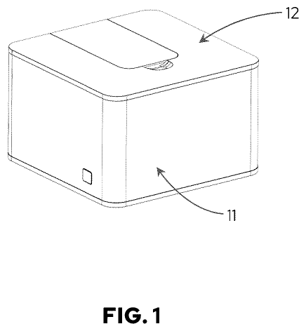

Referring to FIG. 1, the principal system (20) of the multimodal and modular optical apparatus (10) can include a housing (11) defining walls for containing functional components of the system (not shown) and a removable cover (12) that is fitted to the housing (11).

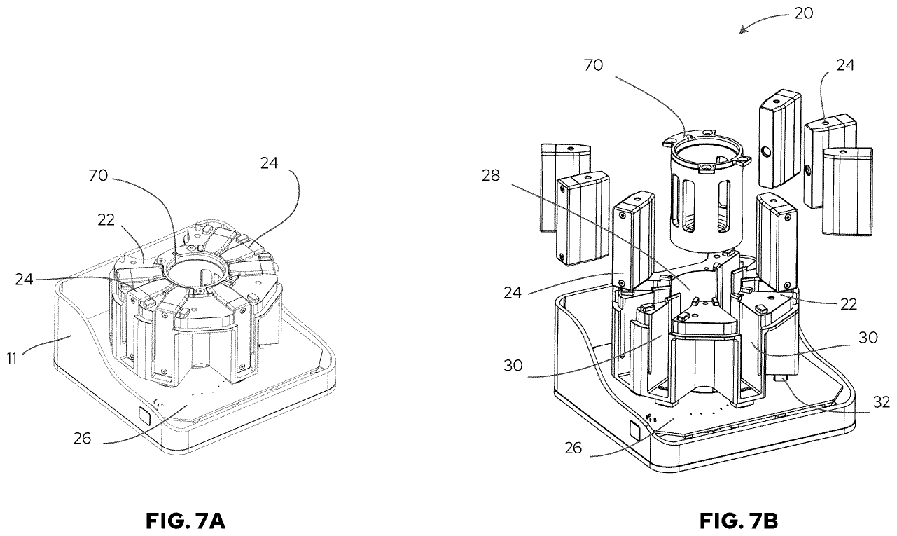

The interior of the principal system of the multimodal and modular optical apparatus is composed of mechanical, optical, electrical and electronic parts and components that ensure operation of the apparatus, along with acquisition and generation of data during operation of the apparatus. As illustrated in FIG. 7A, hidden by the housing (11) of the principal system (20) of the multimodal and modular optical apparatus (10), the principal system (20) includes a main body (22) that is sized and shaped for receiving a material sample (not shown) and at least one optical cartridge (24) for acquiring optical data related to the material sample. The principal system (20) further includes a control and processing unit (26) being connected to at least one cartridge (24) for controlling electronic operation of the apparatus and ensuring data communication. FIG. 7B is an exploded perspective view that illustrates in more detail how the principal system of the multimodal and modular optical apparatus can be constructed. The main body (22) defines a sample receiving space (28) that can be central to the main body and at least one cartridge-receiving space (30) in optical communication with the at least one sample receiving space (28). The sample receiving space (28) and each cartridge-receiving space (30) can be designed as open cavities defined in the main body (22). Each sample receiving space (28) is sized and shaped for reversible insertion of the material sample. In the embodiment shown in FIGS. 7B, the at least one cartridge-receiving space (30) comprises a plurality of the cartridge-receiving spaces (30) for housing one or more removable cartridge(s) (24). The main body (22) can further include a communication connector (32) to connect each cartridge (24), when inserted in a corresponding cartridge-receiving space (30), to the control and processing unit (26), thereby ensuring power supply and data communication between the connected cartridge (24) and the control and processing unit (26). For example, the communication connector (32) can be pins being receivable in complementary sockets, electrical contacts, gold fingers and slots, pogo pins and electrical contact, male and female pin connectors, and other related electrical contact devices.

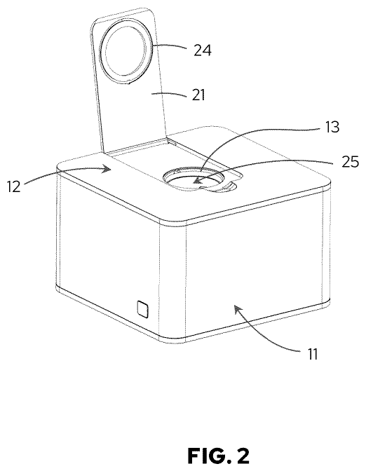

FIG. 2 shows the principal system (20) of the multimodal and modular optical apparatus (10) for acquiring optical data from a material sample to characterize its physical, chemical or biological properties with the cover (12) including a lid (21) that can be moved from a closed position to an open position to provide access to an interior of the principal system for a material sample via an opening (13) defined in the cover (12).

In some implementations, as seen in FIG. 2, the lid (21) can be referred to as an analysis chamber lid (21) giving access to an analysis chamber (25) that is enclosed within the housing (11). The analysis chamber lid (21) is connected to the cover (12) and the cover (12) is connected to the housing (11) of the principal system (20) of the multimodal and modular optical apparatus (10). As shown in FIG. 2, the analysis chamber lid (21) can include a seal (24) protruding from a bottom surface of the lid (21) and matching the opening (13) in the cover (12) to seal the analysis chamber (25). The sealing allows preventing, for example, gasses exchanges between the analysis chamber (25) and an outside environment, heat exchange between the analysis chamber (25) and the outside environment, or light penetration into the analysis chamber (25) from the outside environment of the principal system.

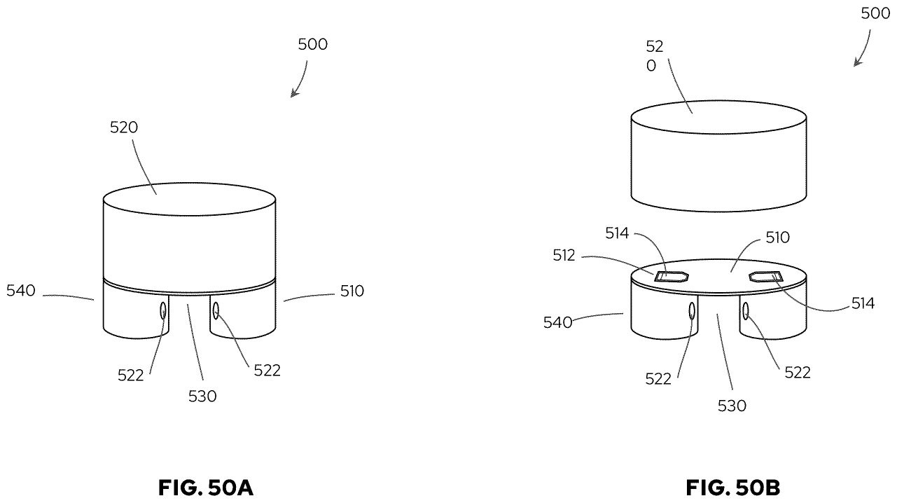

In other implementations, for example referring to the principal system (500) illustrated in FIGS. 50A and 50B, the main body can be a sealed main body (510) allowing at least partial immersion of the principal system (500) into a liquid material to be characterized. The system (500) includes a removable cover (520) that can be positioned in sealing engagement with the main body (510) to seal the cartridge-receiving spaces and any other connectors being accessible from the top surface of the main body (510).

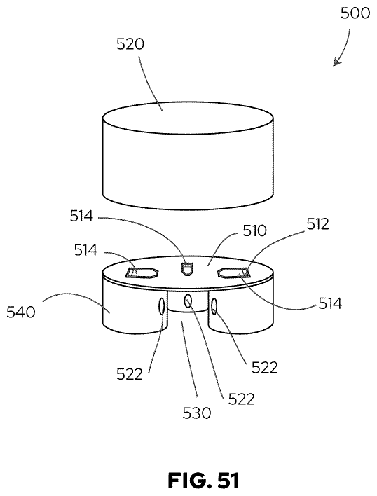

In other implementations, as seen in FIGS. 50A, 50B and 51, the cover (520) can be removed/disconnected from a top surface of the sealed main body (510) to reveal at least one cartridge-receiving space (512), thereby enabling insertion or removal of a cartridge (514) within one or more cartridge-receiving spaces (512). In the implementation illustrated in FIGS. 50A, 50B, 51A and 51B, the sample receiving space is not accessible from a top surface of the main body (510) as per other implementations but rather provided as a recess or gap (530) being defined between protruding elements (540) of the main body (510). More particularly, the illustrated design allows the system (500) to be immersed into a liquid material and perform in situ measurements of any liquid material being present within the region of the gap (530) that serves as the sample receiving space. It should be noted that the design of the main body (510) can differ from the one shown in FIGS. 50A, 50B and 51 as long as the housing can define a space for receiving the liquid material when the main body (510) is immersed into the liquid material.

It is noted that the liquid material that is tested with the immersion testing implementation of the system (500) can also be referred to as a material sample as generally used herein so that other features recited in combination with the term material sample are compatible with the immersion testing implementation of the system (500).

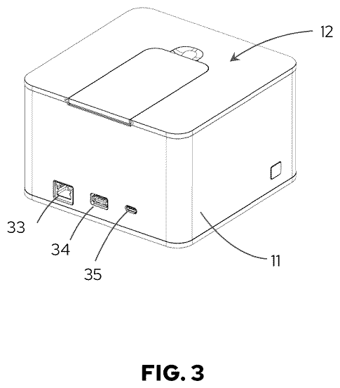

In some implementations, the principal system can include additional multiple connectors providing for at least one of data communication, data storage and power communication. For example, referring to FIG. 3, the principal system (20) can include three connectors (33 to 35) providing communication and power connections. The housing (11) can define apertures giving access to the connectors (33 to 35). More particularly, a first connector (33) can be a network connector (33) allowing connection of the principal system to an internet or intranet network through, for example, an ethernet connection. A second connector (34) can be a storage connector (34) allowing connection of external storage device(s) through, for example, a USB connection to store data acquired and generated by the principal system. A third connector (35) can be a power connector (35) allowing connection of the principal system to a power source or a power adapter through, for example, a USB-C connection.

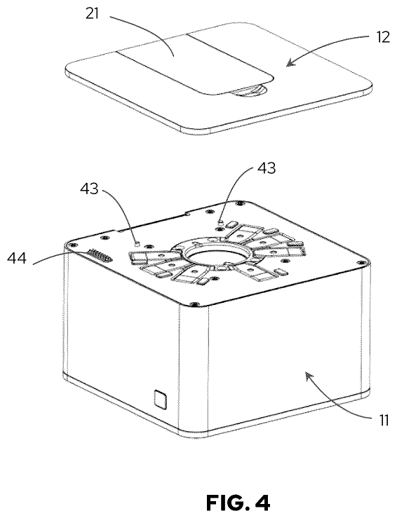

The cover of the principal system of the multimodal and modular optical apparatus may be removed to access the cartridge-receiving space(s) of the main body, thereby enabling insertion or removal of a cartridge within one or more cartridge-receiving space(s). The principal system can include at least one alignment mechanism to ensure alignment and fitting of the cover and housing when closing the principal system. The alignment mechanism can include one or more protrusion(s) and corresponding notch(es) defined in the cover and housing. As illustrated in FIG. 4; the cover (12) of the principal system of the multimodal and modular optical apparatus (10) can be removed from the housing (11) to reveal a plurality of alignment protrusions (43) extending vertically and outwardly from a surface of a top wall of the housing (11).

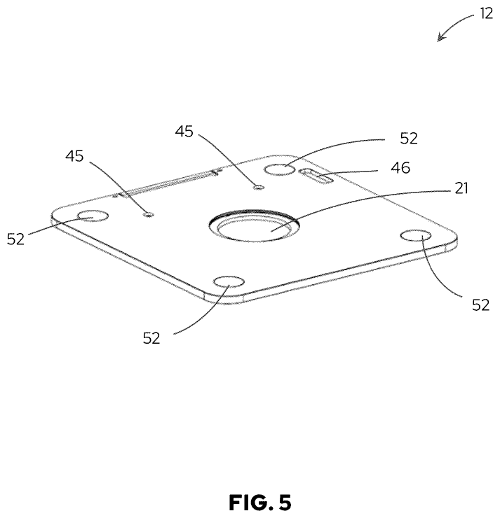

As shown in FIG. 5, the cover (12) can include a plurality of alignment notches (45) being sized and shaped to receive the alignment protrusions of the housing shown in FIG. 4, to ensure the alignment of the cover (12) with the housing of the principal system of the multimodal and modular optical apparatus.

Still referring to FIG. 5, the cover (12) can further include a hooking mechanism (52) to ensure the fixation of the cover (12) to the housing of the apparatus. For example, magnets can be provided in the housing for hooking to the hooking mechanism of the cover. However, any mechanism ensuring reversible securing of the cover to the housing can be used as readily understood by one skilled in the art. The correct and permanent alignment of the cover with the principal system of the multimodal and modular optical apparatus ensures that the lid of the cover is correctly positioned to give access to the sample receiving space of the principal system.

The principal system of the multimodal and modular optical apparatus may be connected to additional functional and/or operational modules being external to the principal system. For example, such additional external functional or operational modules can be connectable from a top or bottom of the principal system to confer additional measurement modalities/functions or additional operations/actions to the apparatus. It should be noted that additional connectors can be provided to provide additional connections to exterior systems including an additional functional/operational module, a display device or another optical measurement apparatus as defined herein. Additional connectors can also be provided within the principal system to ensure communication of power and/or data among components of the principal system as will be further described herein.

It should be noted that the cover can include additional notches and depressions to accommodate protrusions and other elements stemming from a top surface or bottom surface of the housing.

For example, as shown in FIG. 4, the principal system (20) can further include at least one universal connector (44) being accessible from a top surface of the housing (11) to allow connection to an additional top module. Referring to FIG. 5, the cover can include a connector notch (46) for providing room for a connector protruding from a top surface of the housing as seen in FIG. 4.

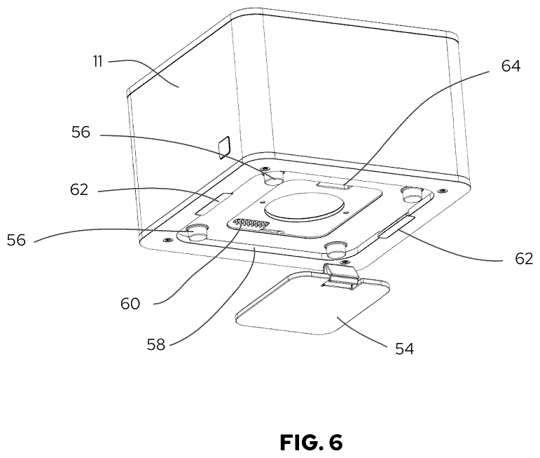

In another example, referring to FIG. 6, the principal system (20) of the multimodal and modular optical apparatus (10) can include a removable access panel (54) that can be fitted within a bottom surface of the housing (11) to hide and protect elements of the system which are formed in the bottom surface of the housing (11) or accessible from a bottom surface of the housing (11). The bottom surface of the housing (11) can for example include two pairs of opposed foot (56) to support the principal system (20) when abutted against a contact surface. The bottom surface of the housing (11) can further define a module-receiving space (58) intended to ensure operational connection of an external additional module with the bottom surface of the housing (11). The bottom surface of the housing (11) can further define an aperture allowing access and connection with another universal connector (60) configured to ensure data communication and, optionally, the power supply between the principal system and the external module (not shown). The access panel (54) can be positioned between the foot (56) of the housing (11) and reversibly lockable within a recess (64) of the housing (11). The access panel (54) can be removed to give access to the module-receiving space (58) and connector (60). The bottom surface of the housing (11) may also include an additional hooking mechanism (62) to maintain the external bottom module (not shown) aligned with the principal system (20).

### Cartridges

Depending on the tested material and the target measurement modality for the apparatus, the cartridges can be used as an electromagnetic receiver and/or emitter. For example, by using removable and interchangeable cartridges, the nature and position of one or more cartridges can be tailored to produce a given light path for a target measurement modality to be operated. The nature of the cartridge can be changed (for example to change an emitter for a receiver or to change a wavelength of the emitter and/or receiver), be moved from one cartridge-receiving space to another to irradiate the material sample and receive electromagnetic radiations from the sample according to different angles and spatial configurations.

The terms “light” and “optical”, and variants and derivatives thereof, refer herein to radiation in any appropriate region of the electromagnetic spectrum. These terms are not limited to visible light, but may also include invisible regions of the electromagnetic spectrum including, without limitation, the infrared (IR) region and the ultra-violet (UV) region.

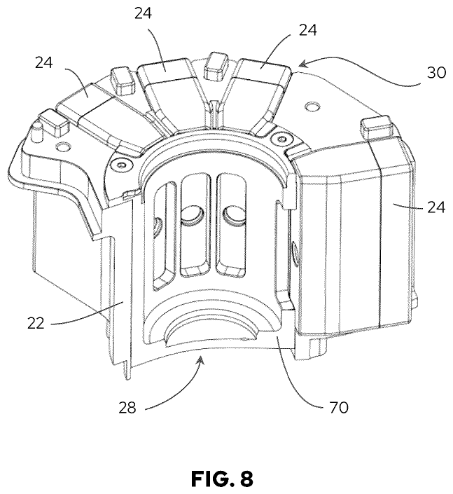

As illustrated in FIG. 8, each cartridge (24) used in the principal system of the multimodal and modular optical apparatus is placed in one of the single or multiple cartridge receiving space(s) (30) of the main body (22). As better seen in FIG. 7B, each cartridge receiving space (30) is in optical communication with the sample receiving space (28), e.g., via an opening or channel defined between the cartridge receiving space (30) and the sample receiving space (28) to allow the electromagnetic radiations to travel therebetween.

Interchangeable cartridges can be connected to the main body and disconnected from the main body. All cartridges comprise optical and electronic arrangements to generate or receive a light beam, a casing to embed the optical and electronic arrangements, at least one optical window defined in the casing to let the light beam go out of the cartridge or to let a light beam enter in the cartridge; and a connector for the bidirectional communication with the control and processing unit of the principal system and to manage the power supply. Optionally, the cartridges can further include an auto alignment mechanism to facilitate adequate installation of the cartridges in the main body of the principal system.

The principal system of the multimodal and modular optical apparatus uses a single or multiple cartridge(s) to emit and/or receive electromagnetic radiations to and/or from a material sample that is positioned in the sample receiving space. A cartridge may be built in a specific way in order to operate different measurement modalities. In some implementations, the at least one cartridge can include an emitter cartridge, a spot emitter cartridge, a linear emitter cartridge, an emitter-receiver cartridge, a receiver cartridge, a spot receiver cartridge, a linear receiver cartridge, a light stimulation cartridge, a Brownian motion cartridge, a raman spectroscopy cartridge, a contactless temperature measurement cartridge, an imaging cartridge, or any combinations thereof. Further details are provided below.

In some implementations, for any cartridge (emitter and/or receiver), the wavelength generated by the cartridge may be adapted to each cartridge.

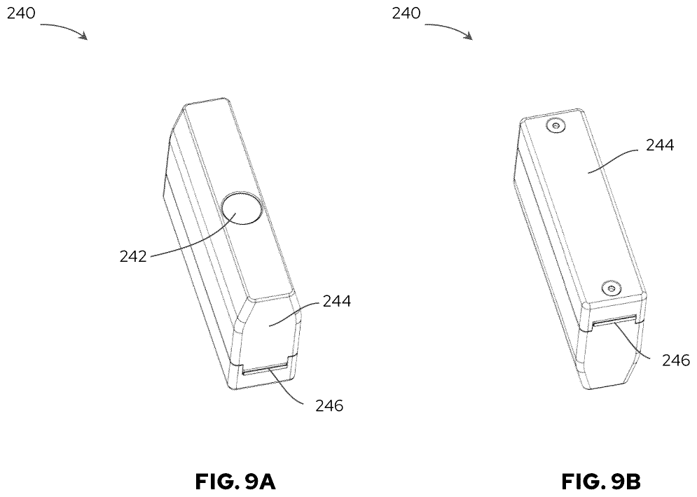

For example, the at least one cartridge can be a spot emitter cartridge comprising a light source that generates a narrow or wide single beam with a spot size from about 0.5 mm diameter to about 2 mm diameter along a light path perpendicular to the longitudinal axis of the sample receiving space. The spot emitter cartridge generates a light beam consisting of a single wavelength from about 200 nm to about 2000 nm, and optionally from 300 nm to about 1000 nm, or a plurality of wavelengths or wavelength ranges (UV range, Vis range, NIR range, IR range or any combinations such as UV-Vis, UV-Vis-NIR, etc.). The spot emitter cartridge generates a light beam with an intensity from about 0 mWatts to about 50 mWatts, and optionally from 0 mWatts to about 10 mWatts. Referring to FIG. 9A, the casing (244) of the spot emitter cartridge (240) includes a light source optical window (242) that allows light radiations generated by the cartridge (240) to be emitted from the cartridge (240). The spot emitter cartridge (240) also includes a cartridge communication connector (246) that allows the cartridge (240) to communicate with the control and processing unit of the principal system of the multimodal and modular optical apparatus. FIG. 9B shows a rear view of the spot emitter cartridge (240) with its casing (244) and the communication connector (246).

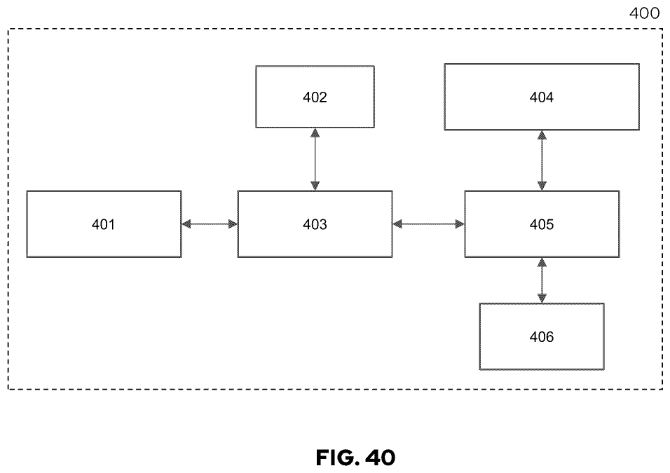

FIG. 40 further schematizes operation of an emitter cartridge (400) that may include a light source (402) to emit electromagnetic radiations of different properties, sensors (401) to perform a plurality of environmental measurements such as temperature, humidity, light, gas, etc., connected to a hardware manager (403). In some implementations, the emitter cartridge (400) may also comprise a processor (405) for the data processing and control, a memory (395) to store configuration data, measurement data, etc. and a communication interface (404) that may be in communication with the control and processing unit (381) of the principal system.

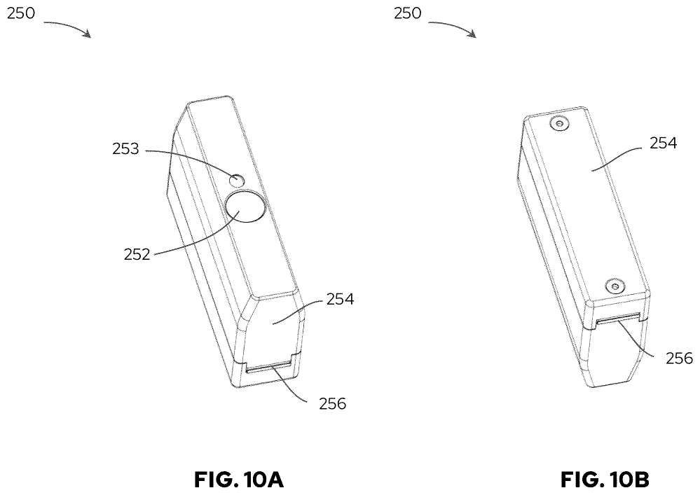

In some implementations, one or more emitter-receiver cartridge(s) may be provided to emit light and receive back-scattered light from the material sample that is analyzed using the principal system of the multimodal and modular optical apparatus. FIGS. 10 B to 11B show two different implementations of the emitter-receiver cartridge. Referring to FIG. 10A, the casing (254) of the emitter-receiver cartridge (250) includes a light source optical window (252) that allows light radiations generated by the cartridge (250) to be emitted and a backscatter optical window (253) that receives the light backscattered by the material sample in order to measure its intensity. The emitter-receiver cartridge (250) also includes a cartridge communication connector (256) that allows the cartridge (250) to communicate with the control and processing unit of the principal system of the multimodal and modular optical apparatus. FIG. 10B shows a rear view of the emitter-receiver cartridge (250) with its casing (254) and the communication connector (256). The emitter-receiver cartridge can be configured to measure the intensity of light over a wide range of wavelengths and be used to measure the spectrum of received light. The emitter-receiver cartridge comprises a light source that generates a narrow or wide single beam with a spot size from about 0.5 mm diameter to about 2 mm diameter, or a line beam with a thickness from about 0.1 mm to about 2 mm and a width of about 2 mm to about 30 mm, along a light path at an angle of 90°±45° with respect to the longitudinal axis of the sample receiving space. The emitter-receiver cartridge generates a light beam consisting of a single wavelength from about 200 nm to about 2000 nm and preferably from 300 nm to about 1000 nm, or a plurality of wavelengths or wavelength ranges (UV range, Vis range, NIR range, IR range or any combinations such as UV-Vis, UV-Vis-NIR, etc.). The emitter-receiver cartridge generates a light beam with an intensity from about 0 mWatts to about 50 mWatts and preferably from 0 mWatts to about 10 mWatts. The emitter-receiver cartridge comprises a backscatter light detector positioned at an angle between about 10° to about 40° with respect to the light path or twice the angle between the angle of the light path and the angle of the longitudinal axis of the analysis chamber.

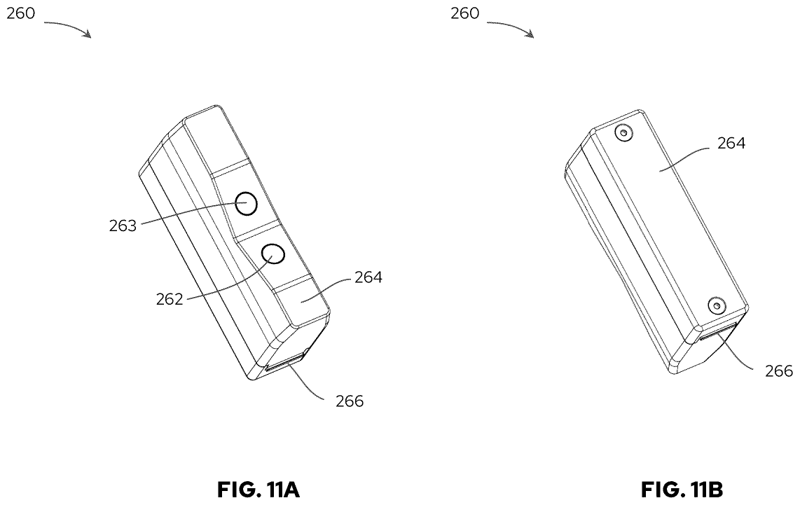

Referring to the other implementation shown in FIG. 11A, the casing (264) of the emitter-receiver cartridge (260) can include two optical windows (262 and 263) that allow light radiations generated by the cartridge (260) to be emitted by one of the optical windows (e.g., 262) and to the light backscattered by the material sample to be received by the other optical window (e.g., 263) in order to measure its intensity. The optical windows (262 and 263) can be built in the casing (264) with an angle in order to optimize the quality of the emitted and received light. The emitter-receiver cartridge (260) also includes a cartridge communication connector (266) that allows the cartridge (260) to communicate with the control and processing unit of the principal system of the multimodal and modular optical apparatus. FIG. 11B shows a rear view of the emitter-receiver cartridge (260) with its casing (264) and the cartridge communication connector (266). In some implementations, the one or more emitter-receiver cartridge(s) can be configured to measure the intensity of light over a wide range of wavelengths and be used to measure the spectrum of received light. The emitter-receiver cartridge comprises a light source that generates a narrow or wide single beam with a spot size from about 0.5 mm diameter to about 2 mm diameter, or a line beam with a thickness from about 0.1 mm to about 2 mm and a width of about 2 mm to about 30 mm, along a light path at an angle of 90°±45° with respect to the longitudinal axis of the analysis chamber. The emitter-receiver cartridge generates a light beam consisting of a single wavelength from about 200 nm to about 2000 nm and preferably from 300 nm to about 1000 nm, or a plurality of wavelengths or wavelength ranges (UV range, Vis range, NIR range, IR range or any combinations such as UV-Vis, UV-Vis-NIR, etc.). The emitter-receiver cartridge generates a light beam with an intensity from about 0 mWatts to about 50 mWatts, and optionally from 0 mWatts to about 10 mWatts. The emitter-receiver cartridge comprises a backscatter light detector positioned at an angle between about 10° to about 40° with respect to the light path or twice the angle between the angle of the light path and the angle of the longitudinal axis of the sample receiving space.

Referring to the immersive testing implementations as shown in FIGS. 50B and 51, the system (500) includes at least one cartridge-receiving space (512), each being configured to receive a cartridge as defined herein (e.g., emitter cartridge, receiver cartridge, emitter-receiver cartridge, or any combinations thereof). For example, the system (500) can include two cartridge-receiving spaces (512). For example, the system (500) can include three cartridge-receiving spaces (512). As per other implementations, the cartridge-receiving spaces (512) are accessible from an opening in the top surface of the sealed main body (510) and can be further defined by inner parts of the sealed main body (not shown). The main body (510) further includes a number of optical windows (522) corresponding to the number of cartridge-receiving spaces (512). For example, in FIG. 50B, at least two optical windows (522) are provided in the protruding elements (540) of the main body (510). Each optical window (522) allows light that is emitted from each inserted cartridge (514) to be transmitted through the corresponding optical window (522) to the liquid material being present within the gap (530) when the main body (510) is immersed in the liquid material. The positioning of the optical windows (522) is dictated by the design of the inserted cartridges in accordance with the configuration allowed by the cartridge-receiving space (not shown). The optical windows (522) of the main body can be referred to as external optical windows (522) and are configured to be aligned with any optical window provided in the casing of a cartridge (514), when inserted. For example, the cartridge(s) (514) may be inserted at an angle along either side of the main body (510) and within a corresponding protruding element (540), aiming to generate light at different angles in a direction of the liquid material being present within the gap (530), and to further measure the resulting light coming from the exposed liquid material at different angles. Multiple optical windows (522) and corresponding protruding elements (540) of the main body (510) can be provided to acquire the desired in situ optical data. FIG. 50B shows an implementation with two optical windows for corresponding cartridges while FIG. 51 shown another implementation with three optical windows for corresponding cartridges.

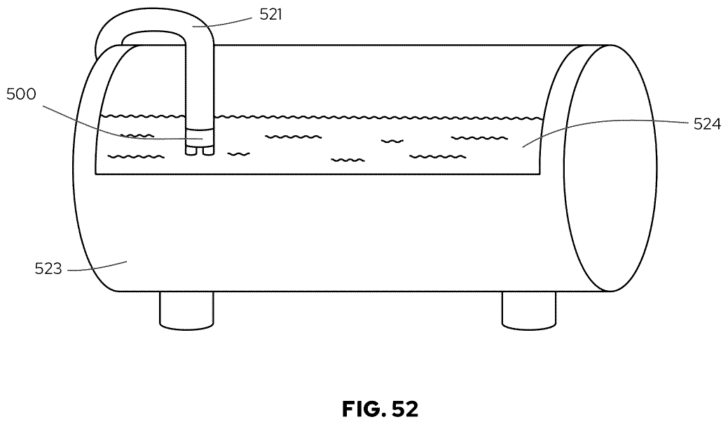

When using the immersive testing implementation of the principal system (500), the main body (510) can be immersed into an industrial or lab scale vat/tank to measure the optical properties of any liquid material being contained in the vat/tank. For example, the immersive testing system (500) may be used to measure the time evolution of the optical properties of a liquid material during a production process in order to monitor and control such a process. FIG. 52 illustrates an immersive testing system (500) being permanently or reversibly connected to an end portion of an elongated arm member (521) extending towards a surface of the liquid material (524) when contained in the vat/tank (523) so as to immerse the connected immersive testing system (500) into the liquid material (524) to be optically tested. Measurements of the immersive testing system (500) can be communicated from the control and processing unit (not shown) of the system (500) to an industrial automatic control system to monitor and adjust production steps.

In some implementations, there is provided a process comprising real time monitoring of at least one parameter characterizing a physical, chemical and/or biological property of a liquid material by measuring optical data from the liquid material using the multimodal and modular optical apparatus as defined herein, with the optical data being correlated to the at least one parameter. For example, there is provided a process comprising monitoring the coagulation of milk in the dairy industry for the production of cheese and yogurt by measuring optical data from the milk using the immersion testing system as defined herein. For example, there is provided a process comprising monitoring the microbiological fermentation in a liquid medium by measuring optical data from the liquid medium using the immersion testing system as defined herein. For example, there is provided a process comprising monitoring of cell proliferation in a liquid medium by measuring optical data from the liquid medium using the immersion testing system as defined herein.

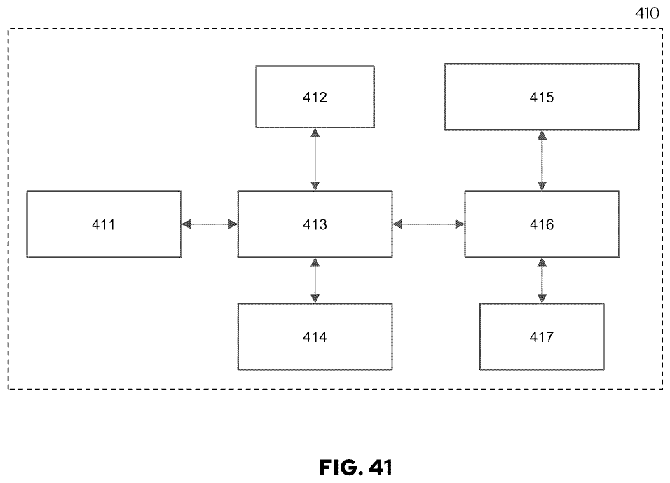

FIG. 41 schematizes operation of the emitter-receiver cartridge (410) that may include a light source (412) to emit electromagnetic radiations of different properties and a light detector (414) to measure electromagnetic radiations of different properties. The emitter-receiver cartridge (410) can also comprise sensors (411) to perform a plurality of environmental measurements such as temperature, humidity, light, gas, etc., connected to a hardware manager (413). In some embodiments, the emitter-receiver cartridge (410) may also comprise a processor (416) for the data processing, a memory (417) to store configuration data, measurement data, etc. and a communication interface (415) that may be in communication with the control and processing unit (381) of the principal system.

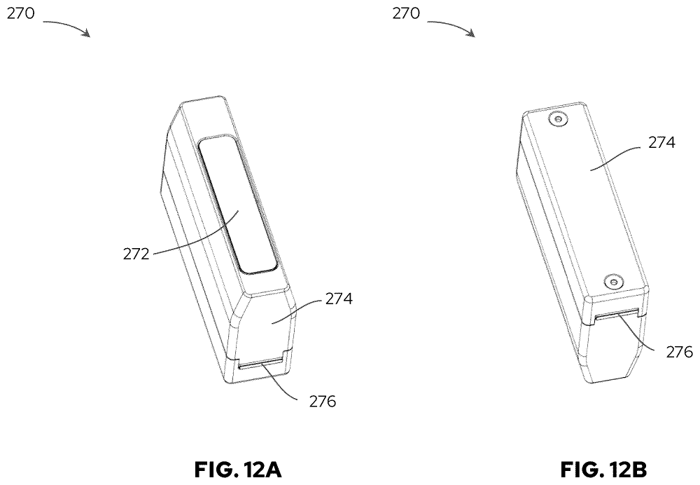

In some implementations, one or more linear emitter cartridge(s) may be provided to emit light following a continuous or discrete line in order to expose a material sample to light in different spatial locations that follow a continuous or discrete line. The linear emitter cartridge comprises a light source that generates a line beam along the longitudinal axis of the analysis chamber generated by single or multiple light sources with or without the combination of a vertical slit, having a length from about 0 mm to about 35 mm, and a width (thickness) from about 0.5 mm to about 10 mm. The linear emitter cartridge generates a light beam consisting of a single wavelength from about 200 nm to about 2000 nm and preferably from 300 nm to about 1000 nm, or a plurality of wavelengths or wavelength ranges (UV range, Vis range, NIR range, IR range or any combinations such as UV-Vis, UV-Vis-NIR, etc.). The linear emitter cartridge generates a light beam with an intensity from about 0 mWatts to about 50 mWatts, and optionally from 0 mWatts to about 10 mWatts. Referring to FIG. 12A, the casing (274) of the linear emitter cartridge (270) includes a linear light source optical window (272) that allows light radiations generated by the cartridge (270) to be emitted. The linear emitter cartridge (270) also includes a communication connector (276) that allows the cartridge (270) to communicate with the control and processing unit of the principal system of the multimodal and modular optical apparatus. FIG. 12B shows a rear view of the linear emitter cartridge (270) with its casing (274) and the communication connector (276).

In some implementations, the one or more cartridge(s) can be configured in order to receive and measure the intensity of light that traveled across a material sample. The wavelength range of measurement of the spot receiver cartridge may be adapted to each cartridge following the target application. For example, a spot receiver cartridge can be configured to measure the intensity of light over a wide range of wavelengths and be used to measure the spectrum of received light. The spot receiver cartridge comprises a light detector sensitive to light having a single wavelength from about 200 nm to about 2000 nm, and optionally from 300 nm to about 1000 nm, or a plurality of wavelength ranges (UV range, Vis range, NIR range, IR range or any combinations such as UV-Vis, UV-Vis-NIR, etc.). In another example, a receiver cartridge can be configured to operate as a spectrometry receiver cartridge. The spectrometry receiver cartridge comprises a diffraction grating and an array detector that separates the received light into about 1000 to about 4000 component wavelengths from about 200 nm to about 2000 nm, and optionally from 300 nm to about 1000 nm, or from a plurality of wavelengths or wavelength ranges (UV range, Vis range, NIR range, IR range or any combinations such as UV-Vis, UV-Vis-NIR, etc.).

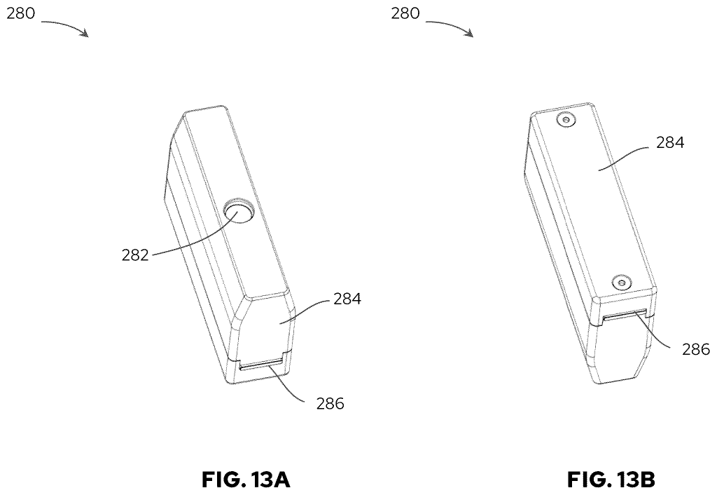

FIG. 13A shows a spot receiver cartridge (280) having a casing (284) which includes a receiver optical window (282) that allows light radiations to be received by the cartridge (280). The spot receiver cartridge (280) also includes a communication connector (286) that allows the cartridge (280) to communicate with the control and processing unit of the principal system of the multimodal and modular optical apparatus. FIG. 13B shows a rear view of the spot receiver cartridge (280) with the casing (284) and the communication connector (286).

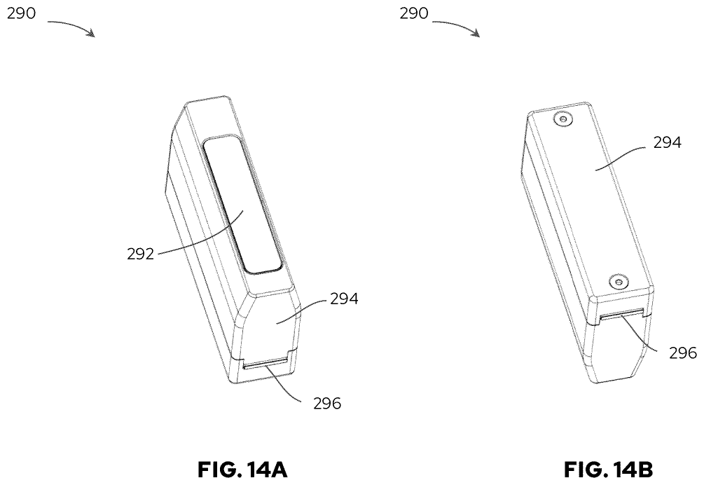

In some implementations, the one or more cartridge(s) can be configured to receive light following a continuous or discrete line in order to measure the intensity of a light beam or a spot light that crossed a material sample. For example, the cartridge can be a linear receiver cartridge that is configured to measure the intensity of light over a wide range of wavelengths and be used to measure the spectrum of received light. The linear receiver cartridge comprises an array detector of about 1000 to 4000 elements positioned parallel to the longitudinal axis of the analysis chamber that measures the received light as function of the longitudinal axis of the analysis chamber. The linear receiver cartridge could comprise a vertical slit between the receiver optical window and the array detector. Referring to FIG. 14A, the casing (294) of the linear receiver cartridge (290) includes a linear receiver optical window (292) that allows light radiations to be received by the cartridge over a continuous or discrete line. The linear receiver cartridge (290) also includes a communication connector (296) that allows the cartridge (290) to communicate with the control and processing unit of the principal system of the multimodal and modular optical apparatus. FIG. 14B shows a rear view of the linear emitter cartridge (290) with its casing (294) and the communication connector (296).

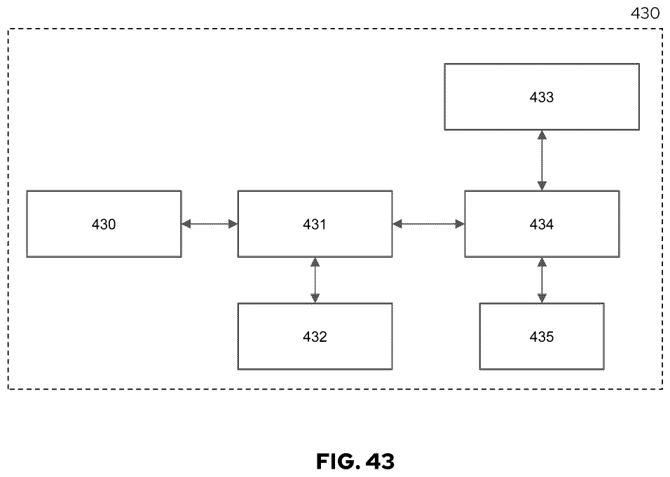

FIG. 43 illustrates a receiver cartridge (430) that may include a light detector (432) to measure electromagnetic radiations of different properties and sensors (430) to perform a plurality of environmental measurements such as temperature, humidity, light, gas, etc. The sensors (430) communicate with a hardware manager (431). In some implementations, the receiver cartridge (430) may also comprise a processor (434) for the data processing, a memory (435) to store configuration data, measurement data, etc. and a communication interface (433) that may be in communication with the control and processing unit (381) of the principal system.

Other possible cartridges that can be connected to the main body of the principal system of the multimodal and modular optical apparatus include:
Light stimulation cartridge: A cartridge comprising a light source and optical arrangements to generate light with wider power range, wider wavelength ranges, narrowed wavelength range, higher intensity, etc., that stimulate the sample material during or between measurements.
Brownian motion cartridges:
Emitter cartridge: Cartridge comprising a laser source to generate high-intensity light in the sample
Receiver cartridge: Cartridge comprising a light sensor to measure the resulting particle motion and a dedicated acquisition and processing unit to acquire and process the signals.
Raman spectroscopy cartridges:
Emitter cartridge: Cartridge comprising a laser source and optical arrangements (lens, filters, etc.) to generate high-intensity light transmitted to the sample.
Receiver cartridge: Cartridge comprising a light receiver sensor arranged around the sample container to receive the Raman scattering and transmitted to a spectrometer for the extraction of the Raman spectra.
Contactless temperature measurement cartridge: Cartridge comprising a sensor such as radiation thermometers, thermal imagers, infrared temperature sensors or any contactless temperature sensors, to measure the temperature of an object without contact.
Imaging cartridges:
Emitter cartridge: Cartridge comprising a light source and optical arrangements (lens, filters, etc.) as an illuminator to generate light transmitted to the sample to image.
Receiver cartridge: Cartridge comprising an optical arrangement and a digital camera to record the image of the sample.

The principal system of the multimodal and modular optical apparatus can include additional mechanical, optical, electrical and electronic components to ensure adequate operation of the apparatus.

### Analysis Chamber

For example, as illustrated in FIG. 7A, the principal system (20) can include an analysis chamber (70) that is positionable in the sample receiving space (28) of the main body (22) of the principal system (20). The analysis chamber (70) can contain the material sample analyzed by the apparatus and facilitate precise positioning of the material sample with respect to the at least one cartridge and generated light path.

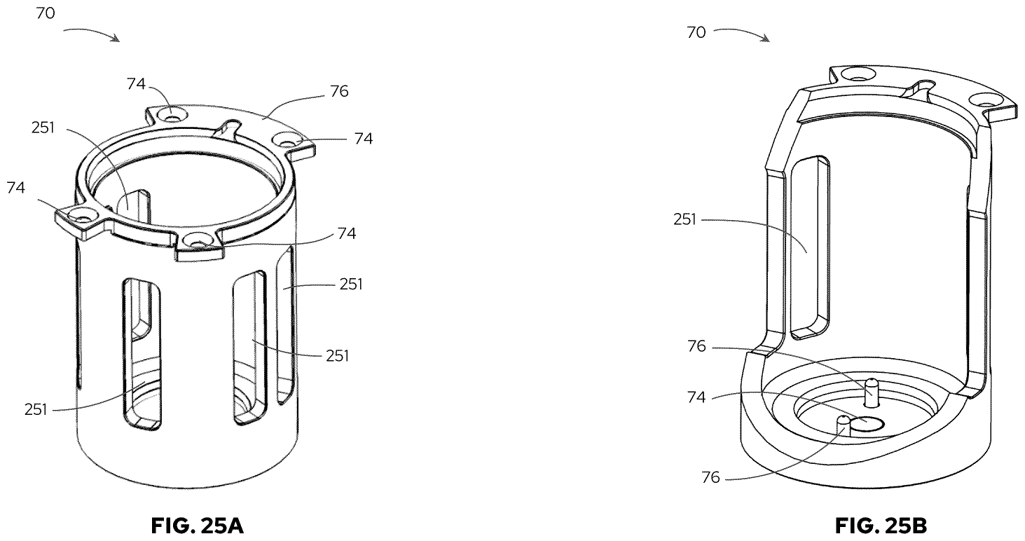

Referring to FIGS. 7A and 7B, the interchangeable single chamber analysis chamber (70) can be easily inserted in the central sample receiving space (28) of the main body (22) of the principal system (20) of the multimodal and modular optical apparatus (10) and can be removed from the main body (22) when needed. The analysis chamber can include one or more wall(s) defining an open basket that can hold the material sample and allow insertion of the material sample from a top of the analysis chamber. The analysis chamber is further designed to allow electromagnetic radiations to reach the material sample when inserted therein. As seen in FIGS. 7B and 25A, the analysis chamber can for example comprise at least one opening (72), that can be multiple openings, in a wall of the analysis chamber to allow radiations transmission between the material sample located in the sample receiving space (28) and the cartridges located in the cartridge receiving spaces (30). In some implementations, each opening in the analysis chamber can be an optical window.

The analysis chamber is thus positioned such that each opening or optical window of the analysis chamber is facing a cartridge receiving space of the main body of the principal system. In some implementations, to facilitate the positioning and reversible securing of the analysis chamber in the sample receiving space of the main body, the analysis chamber can include a hooking mechanism for operational/mechanical connection with the main body. For example, the hooking mechanism can include a snap-fit hooking mechanism, a magnetic hooking mechanism or a bolting mechanism. Referring to FIG. 25A, the hooking mechanism (74) includes a recess for receiving a bolt. For example, referring to FIG. 25B, the sample analysis chamber can further comprise an auto alignment mechanism (76) and/or a centering mechanism that facilitates the insertion of the analysis chamber inside the main body of the principal system.

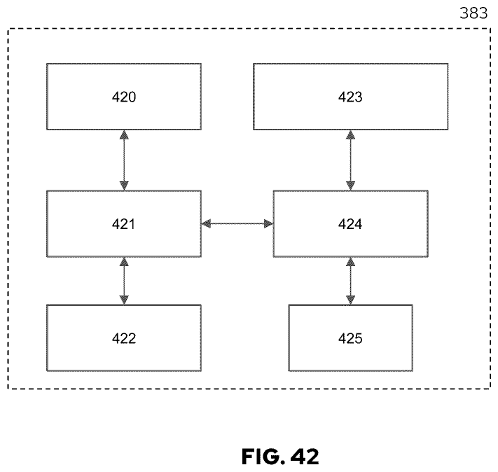

FIG. 42 illustrates operation of the analysis chamber (383) that may further include actuators (420) to perform automatic operation such as handling, rotating, scanning, etc. The analysis chamber (383) also may include sensors (422) to perform a plurality of measurements such as temperature, images, light, humidity, gas, etc. The analysis chamber (383) is also composed of a hardware manager (421) to parametrize, configure and control the actuators (420) and the sensors (422). In some implementations, the analysis chamber (383) may also comprise a processor (424) for data processing, a memory (425) to store data and configuration and a communication interface (423) that may be in communication with the control and processing unit (381) of the principal system.

The principal system of the multimodal and modular optical apparatus can operate optical measurements of material samples contained in different containers that have different volumes. Depending on the target measurement modality and the available volume of material, a specific sample container may be selected and inserted in the sample receiving space to operate in the principal system of the multimodal and modular optical apparatus.

In an embodiment where the sample receiving space is of the main body is configured to receive an analysis chamber as seen in FIG. 7B, the principal system can further include a sample container adapter that is reversibly insertable in the analysis chamber to facilitate precise positioning of a sample container containing the material sample to be analyzed by the apparatus. A single sample analysis chamber can host one sample container adapter for positioning thereof in the light path generated from at least one cartridge. For example, the sample container can be selected from a cuvette, a vial, and a PCR-tube.

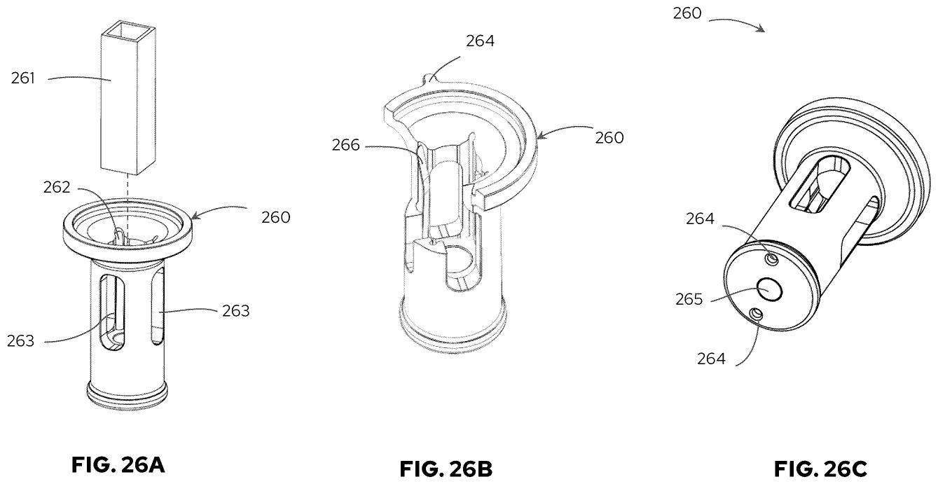

FIG. 26A, FIG. 26B and FIG. 26C show a cuvette sample container adapter (260) that is insertable into the analysis chamber of the apparatus and that can host a cuvette (261). For example, the cuvette can have a volume ranging from 50 μL to 3000 μL. The cuvette is introduced in a recess (262) of the container adapter (260). The container adapter (260) contains openings (263) that allow light beams passing through the container. As better seen in FIG. 26B, the container adapter (260) may include a spring (266) that applies a force on the sample container (not shown in FIG. 26B) in order to maintain its positioning inside the adapter (260). Referring to FIGS. 26B and 26C, the container adapter (260) contains an alignment mechanism (264) and a hooking mechanism (265) to position the sample container adapter correctly inside the analysis chamber and further secure it. For example, the alignment mechanism (264) is a pin-and-hole mechanism, wherein the holes are formed in a lower surface of the adapter and the corresponding pins are provided on an inner surface of the analysis chamber. For example, the hooking mechanism (265) can include magnets.

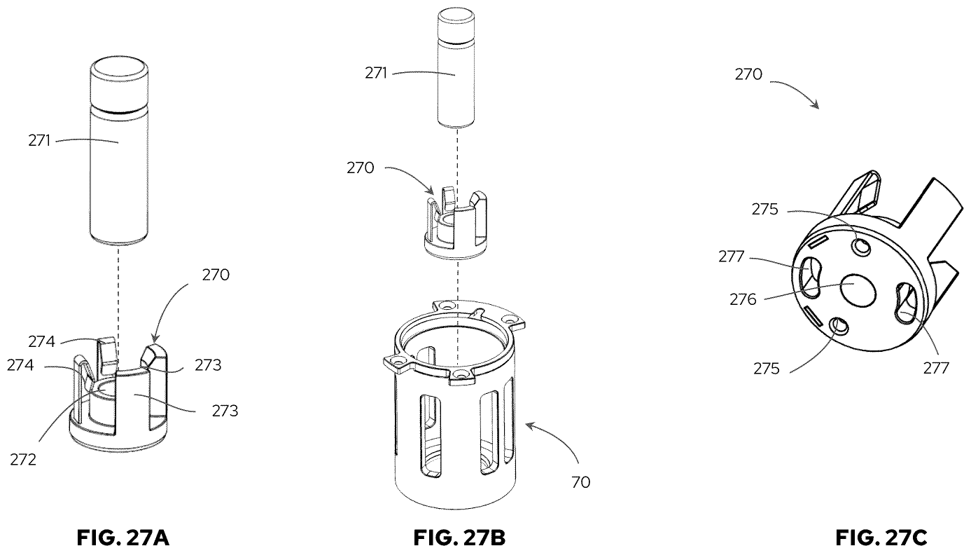

FIG. 27A shows a vial sample container adapter (270) to host a cylindrical vial (271). Optionally, the cylindrical vial can have a volume ranging from 1 mL to 100 mL. The container adapter (270) can include a container recess (272), side stoppers (273) and a spring (274) to precisely guide, locate and maintain the vial sample container (271) in the desired position inside the analysis chamber. Presented in FIG. 27B, the vial sample container (271) is hosted by the vial sample container adapter (270), and the latter is hosted by the analysis chamber (70).

As shown in FIG. 27C, the sample container adapter (270) can contain an alignment mechanism (275) and a hooking mechanism (276) to facilitate position and securing of the adapter (270) with respect to the analysis chamber. The sample container adapter (270) may also contain one or more air opening(s) (277) to allow air circulation inside the analysis chamber in order, for example, to adjust a temperature inside the analysis chamber.

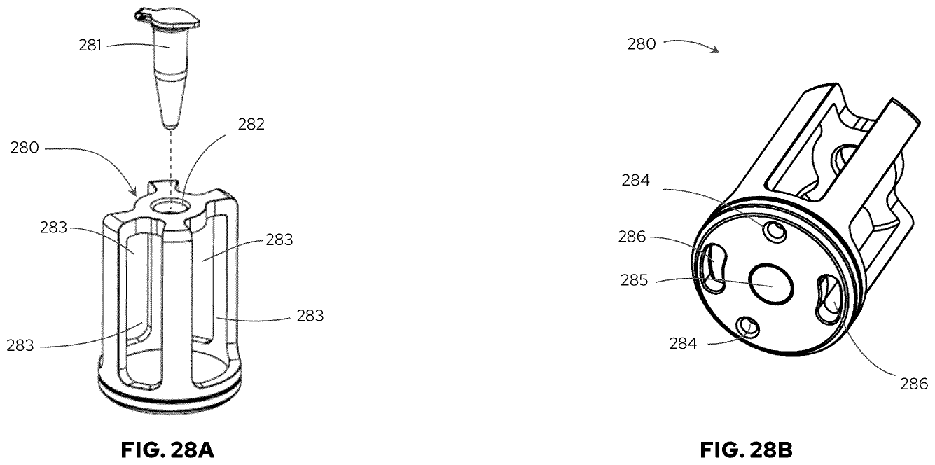

FIG. 28A shows a sample container adapter (280) that is configured to host a PCR-tube (281). Optionally, the PCR-tube can have a volume ranging from 100 μL to 500 μL. The sample container adapter (280) can define a recess (282) and openings (283) that allow light beams to pass through the PCR-tube sample container (281). As illustrated in FIG. 28B, the sample container adapter (280) can further include an alignment mechanism (284) and a hooking mechanism (285) to position the sample container adapter correctly inside the analysis chamber and further secure it. As better seen in FIG. 28B, the sample container adapter (280) may also include one or more air opening(s) (286) to allow air circulation inside the analysis chamber in order, for example, to adjust a temperature inside the analysis chamber.

In some implementations, the analysis chamber can be configured to allow exposure of the material sample to various thermal conditions, in addition to the light path generated upon operation of the cartridges. The analysis chamber can also be configured to actuate exposure of the material sample to the various thermal conditions and communicate the data resulting from such exposure to the control and processing unit of the principal system. When thermal data can be generated from the analysis chamber, the analysis chamber can be referred to as a thermo-optical analysis chamber. Sample container adapters can be hosted in the thermo-optical analysis chamber that is regulated in temperature in order to control the temperature of the material sample being tested by the principal system of the multimodal and modular optical apparatus.

For example, the thermo-optical analysis chamber can provide heat radiations by comprising:
heating elements inside or on a wall surface of the analysis chamber wall working as a radiant heater, and
a secondary control and processing unit to regulate the temperature and to communicate with the control and processing unit of the principal system via an onboard connector.

In another example, the thermo-optical analysis chamber can provide temperature regulated forced convection by comprising:
a double wall to transfer an input air flow having a regulated temperature inside the analysis chamber and extract an output air flow from the analysis chamber, at least one air flow opening defining in a wall of the analysis chamber for circulating the input air flow and output air flow in and out of the analysis chamber via forced convection, and
a secondary control and processing unit to communicate with the control and processing unit of the principal system using an onboard connector.

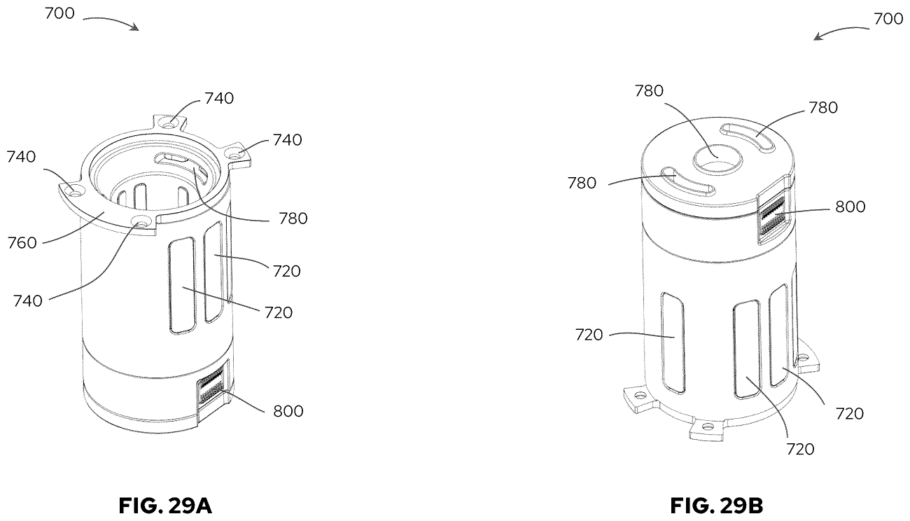

FIG. 29A and FIG. 29B show the thermo-optical analysis chamber (700) that contains hooking mechanisms (740) and alignment mechanisms (760) that ensure the precise positioning of the thermo-optical analysis chamber inside the sample receiving space of the main body of the principal system of the multimodal and modular optical apparatus. The thermo-optical analysis chamber (700) contains optical windows (720) that allow light beams to pass through the thermo-optical analysis chamber (700) and through the sample container (not shown) when inserted in the chamber (700), for example via an optional container adapter (not shown). The thermo-optical analysis chamber (700) further includes an air flow opening (780) to allow input/output air flow circulation of thermally regulated air inside the analysis chamber when used for temperature regulated forced convection. The thermo-optical analysis chamber (700) further comprises a chamber connector (800) to ensure communication with the control and processing unit of the principal system of the multimodal and modular optical apparatus.

It should be noted that, depending on the configuration of the analysis chamber and the target measurement modality, one or more additional modules may need to be connected to the principal system to achieve the target measurement modality. For example, if thermal and optical data is to be recovered from the material sample via the thermo-optical analysis chamber that provides a temperature regulated forced convection, the principal system is to be coupled with an additional module, referred to as a thermal module, so as to be supplied with the regulated temperature air flow and to expel the output air flow to the thermal module. For example, the temperature of the input air flow coming from the thermal module can be regulated by the thermal module.

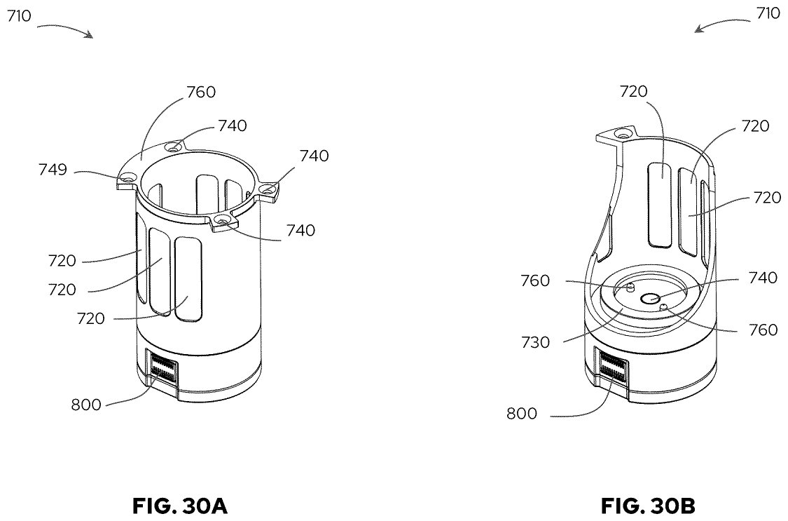

In other implementations, the analysis chamber can be configured to rotate the material sample with respect to the longitudinal axis of the sample receiving space, thereby being referred to as a rotating platform analysis chamber. As illustrated in FIG. 30A and FIG. 30B, the rotating platform analysis chamber (710) comprises a rotary actuator (not shown) aiming to rotate a rotating platform (730) supporting the sample container, e.g., via the sample container adapter. Rotation of the sample container is advantageous to align the light path with a center of the sample container or to position the light path at different locations along a circumference of the sample container.

The rotating platform analysis chamber in combination with a sample container adapter hosting a single sample container can be used to rotate the sample container around its longitudinal axis to perform a plurality of measurements. Optionally, the rotating platform analysis chamber may further comprise heating elements inside or on the surface of the chamber wall working as a radiant heater. The rotating platform analysis chamber further comprises a secondary control and processing unit to control the rotary actuator and to communicate with the control and processing unit of the principal system. Referring to FIGS. 30A and 30B, the communication can be ensured by the onboard chamber connector (800). The rotating platform analysis chamber (710) can include hooking mechanisms (740) and alignment mechanisms (760) that ensure the precise positioning of the rotating platform analysis chamber (710) inside the sample receiving space of the main body of the principal system of the multimodal and modular optical apparatus. The rotating platform analysis chamber (710) further comprises one or more optical window(s) (720) that allow light beams to pass through the chamber and the sample container.

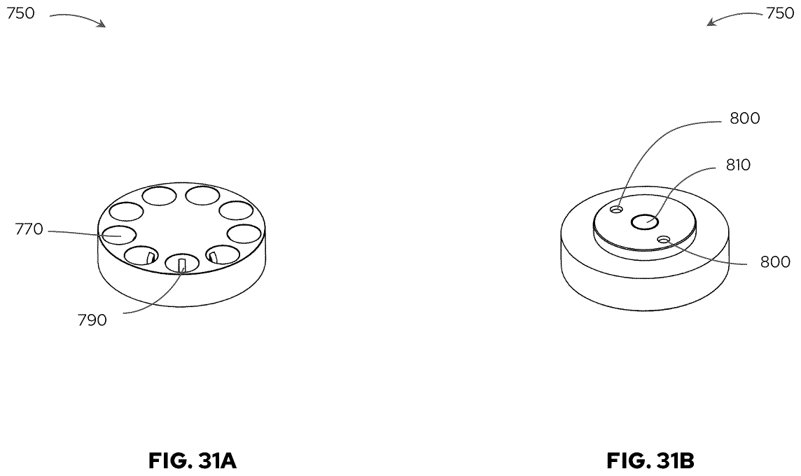

In some implementations, the rotating platform analysis chamber can include a carousel adapter comprising at least one recess that is sized and shaped to host a sample container. The at least one recess is arranged along a circle that is centered with respect to the rotating platform analysis chamber. As illustrated in FIG. 31A and FIG. 31B, the carousel adapter (750) defines a plurality of recesses (770) to host multiple sample containers. The carousel adapter (750) can further comprise multiple springs (790), each spring (790) being encased in a corresponding recess (770) to maintain a position of the sample container (not shown). Referring to FIG. 31B, the carousel adapter (750) can also include at least one alignment mechanism (800) and at least one hooking mechanism (810) that ensure the adequate positioning of the carousel adapter onto the rotating platform of the analysis chamber.

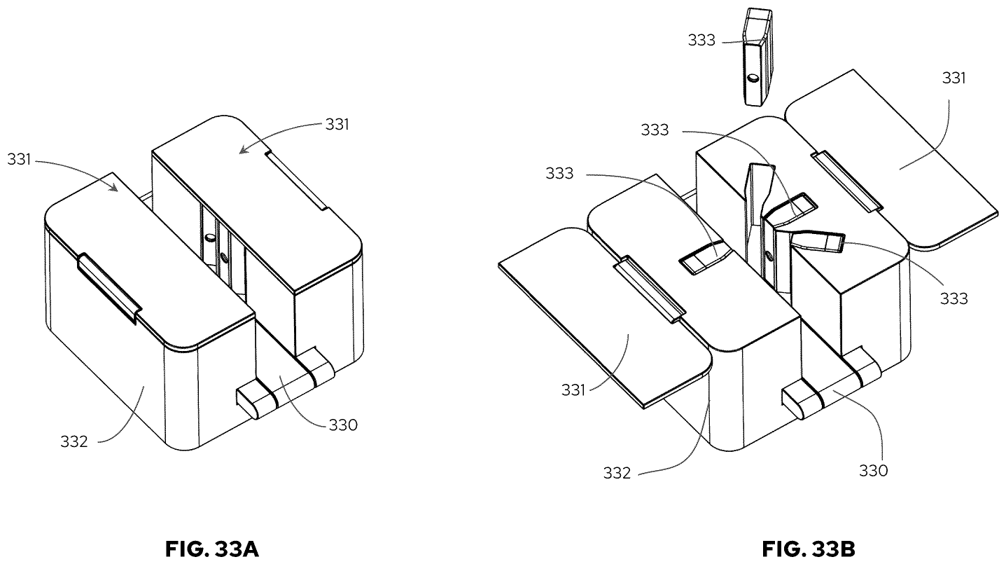

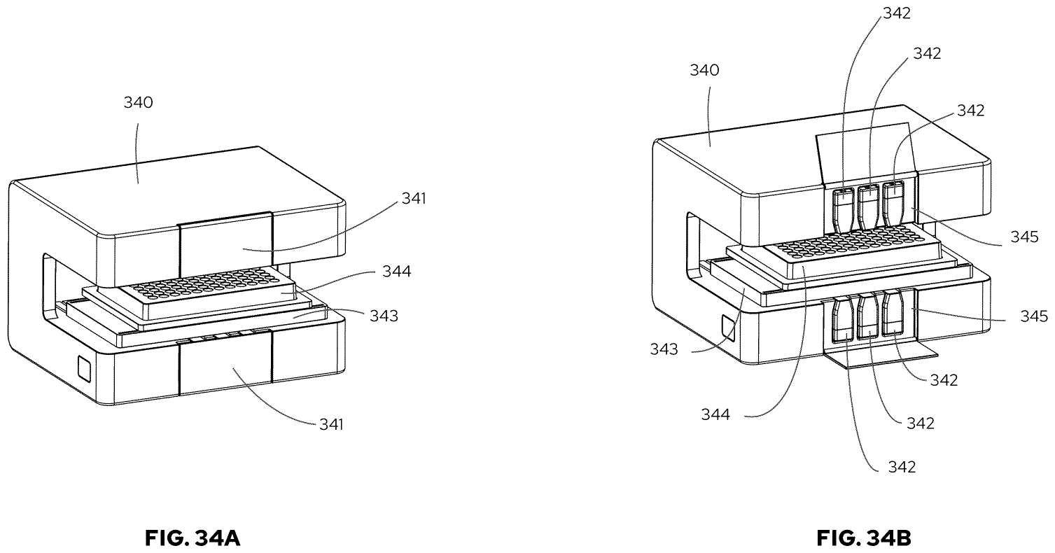

According to two other embodiments of the principal system, the main body, housing and cover can be configured differently from what is shown in FIGS. 1 to 8 to define a sample receiving space allowing conveying of the material sample with respect to the one or more cartridges (see FIGS. 33A and 33B), or scanning of multiple material samples that are positioned in a grid-like fashion (see FIGS. 34A and 34B).

In an embodiment, the principal system can include a conveyor being positioned along the sample receiving space to convey sample materials along the one or more cartridge(s) in order to be tested/measured. The principal system can be referred to as an automatic conveyor handling platform. FIG. 33A and FIG. 33B illustrate the automatic conveyor handling platform (principal system) that integrates a conveyor (330) that can convey material samples in front of the light path, a cover (331) that allows access to the at least one cartridge receiving space of the main body (332) in order to insert and remove at least one cartridge (333) therefrom. The automatic conveyor handling platform further comprises at least one cartridge as described herein (e.g., emitter cartridge, receiver cartridge, emitter-receiver cartridge, or any combinations thereof) disposed in the cartridge receiving space(s) of the main body (332). For example, the cartridge(s) (333) can be disposed at an angle along either side of the conveyor (330), aiming to generate light in the direction of the sample container at different angles and to measure the resulting light coming from the material sample at different angles.

In another embodiment, the main body of the principal system of the multimodal and modular optical apparatus can be configured to operate as an Automatic scanning platform including a positioning platform, such as multi-axis translation stages (e.g., horizontal, vertical, rotation, etc.), that supports a sample container. The principal system further comprises at least one sample container such as a well plate, Petri dish, vial, cuvette, PCR-tube, or any combinations thereof. The system can further include at least one moving or static emitter cartridge, or at least one moving or static emitter-receiver cartridge, disposed in an upper and/or lower part of the main body to generate light towards the sample receiving space in the direction of the sample container(s) on the positioning platform; a moving or static receiver (detector) cartridge, but preferably a set of cartridges disposed in the upper and/or lower part of the main body to measure the resulting light coming from the sample container(s); and a cover that gives access to cartridge receiving space(s) in the main body for insertion and/or removal of the cartridges. FIGS. 34A and 34B illustrate the principal system being an automatic scanning platform composed of a housing (340) and a cover (341) being formed of an upper cover portion and a lower cover portion that, when open, give access to cartridges (342) when inserted in the cartridge receiving spaces of the main body (345). The positioning platform (343) can receive multiple sample containers (344) in different positions to be tested by the principal system of the multimodal and modular optical apparatus.

### Modules

In some embodiments, the multimodal and modular apparatus comprises at least one module that is reversibly securable to the principal system of the apparatus to confer to the apparatus additional functional and/or operational capacities. It should be noted that the at least one module is said to be connected to the principal system and such connection can be at least one of mechanical, electrical and electronics. Mechanical connection is used for securing, e.g., by stacking, the module onto an upper or lower side of the principal system.

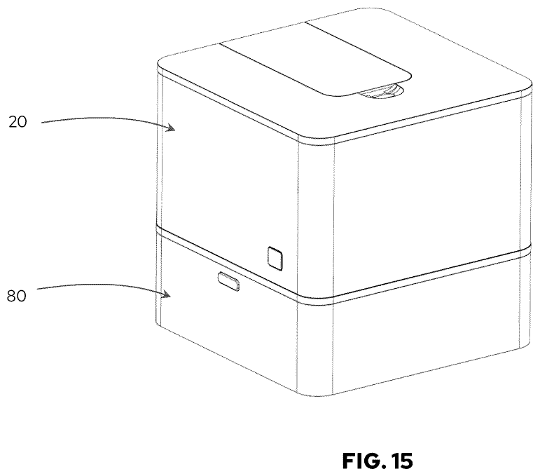

FIG. 15 shows the principal system (20) of the multimodal and modular optical apparatus (10) for acquiring optical and/or thermal data from a material sample to characterize its physical, chemical or biological properties being connected, in this example from a bottom surface, to an external module (80).

Electrical and/or electronic connection of the at least one module to the principal system can be ensured by a universal connector allowing power supply and/or bidirectional data communication with the control and processing unit of the principal system. The at least one additional module generally further includes a hooking mechanism (e.g., snap-fit hooking mechanism, magnetic hooking mechanism, and bolting mechanism) for securing the module to the principal system and a button to release the hooking mechanism from the principal system. Optionally, each module can also include an auto alignment mechanism to be easily abutted against a top surface or bottom surface of the housing of the principal system to ensure adequate (mechanical, electronic and electrical) connection therebetween.

For example, the at least one module of the multimodal and modular optical apparatus include a thermal module, a battery module, a display module, an automatic platform module, a multi-identification module, a carousel dispensing module, an imaging module, a liquid circulation module, a gas injection module, a drop analysis module, a sensor module, a light stimulation module, or any combinations thereof.

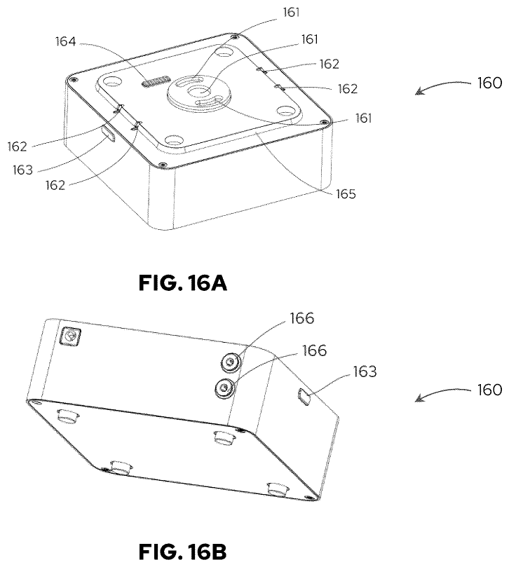

In some implementations, the multimodal and modular optical apparatus includes the thermal module being connected to the bottom universal connector of the principal system. More particularly, the thermal module comprises an outlet for releasing the regulated temperature air flow going to the sample receiving space (and optional analysis chamber) and an inlet for receiving the air flow coming from the sample receiving space (and optional analysis chamber). The thermal module further comprises a heating and/or cooling element used to regulate the temperature of the air flow being supplied into the sample receiving space (and optional analysis chamber). The thermal module further comprises a gas inlet and a gas outlet to respectively supply and release external purge gas (e.g., dry air, Nitrogen, etc.) for mixing with the circulating air volume injected in the sample receiving space. FIG. 16A is a front view of an example thermal module (160) that can be connected to the principal system of the multimodal and modular optical apparatus. The thermal module (160) contains at least one air flow opening (161) positioned to allow the thermally regulated air flow produced by the thermal module to be transmitted to the principal system and the resulting air flow to be recycled back to the thermal module from the principal system. The hooking mechanism (162) of the thermal module includes multiple notches that are complementary to another hooking mechanism of the principal system (not shown) to ensure an adequate connection between the thermal module and the principal system. The release button (163) of the thermal module can be positioned on a side surface of the module to be easily accessible to a user for disconnecting the hooking mechanism (162) from the principal system. The thermal module (160) further includes a universal connector (164) that ensures data communication with the apparatus and, optionally, the power supply. The thermal module may also include an alignment mechanism (165) to ensure the right alignment of the module with the apparatus. FIG. 16B shows a rear view of the thermal module (160) that can be connected to the principal system of the multimodal and modular optical apparatus via the hooking mechanism (162) and the universal connector (164). The release button (163) of the thermal module allows the disconnection (mechanical, electrical and electronic) of the thermal module from the principal system of the multimodal and modular optical apparatus. The thermal module (160) can optionally further comprise gas inlet/outlet connectors (166) allowing the injection and circulation of a gas or a mixture of gas, for example an inert gas or a mixture of inert gas, into the apparatus via the thermal module.

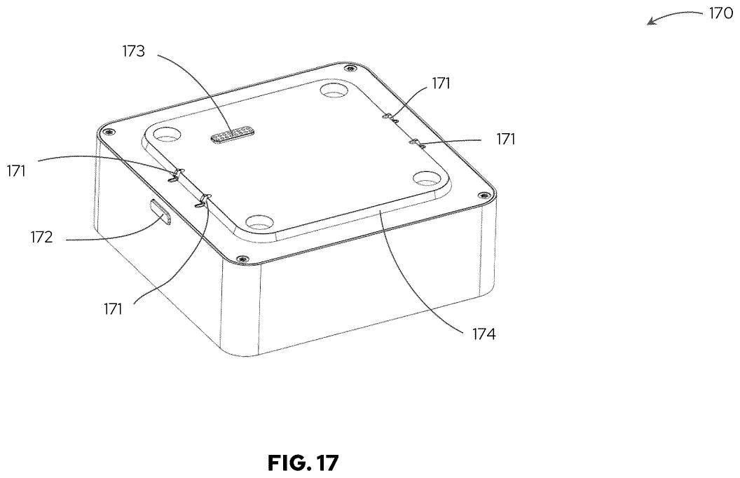

In some implementations, the multimodal and modular optical apparatus includes the battery module being connected to the bottom universal connector of the principal system. For example, the battery module can be connected to the principal system of the multimodal and modular optical apparatus in order to provide power to the apparatus during its operation. The battery module provides, through the universal connector, the power supply to the principal system and the top module. The battery module delivers an energy of up to 400 watt-hour, preferably of about 100 watt-hour. When the principal system is combined with the battery module, the apparatus can be defined as a portable device. FIG. 17A is a view of the battery module (170) that can be connected to the principal system of the multimodal and modular optical apparatus. As per the thermal module described above, the battery module (170) includes a hooking mechanism (171) to ensure an adequate connection between the battery module and the apparatus, and a release button (172) to disconnect the battery module from the apparatus. The battery module also includes a universal connector (173) that ensures communication (electronic and optionally electrical) with the principal system. The battery module may also include an alignment mechanism (174) to ensure the right alignment of the module with the principal system upon connection.

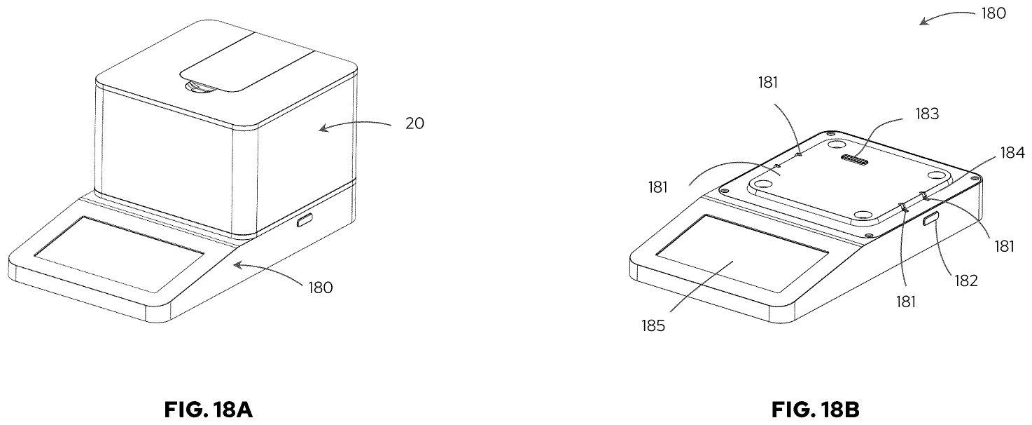

In some implementations, the multimodal and modular optical apparatus includes the display module being connected to the bottom universal connector of the principal system. The display module can be connected to the principal system of the multimodal and modular optical apparatus in order to display data, status information or any relevant information to the user through a user interface. FIG. 18A is a view of a display module (180) connected to the principal system (20) of the multimodal and modular optical apparatus (10). The display module comprises a display device selected from a Liquid Crystal display (LCD), an Organic light-emitting diodes (OLED) display, a Field emission display (FEDs), and a Single emission display (SEDs). The display module communicates with the principal system and is powered through the universal connector. The display module comprises a touch or non-touch panel graphical user interface (GUI) to operate and control the multimodal and modular optical apparatus and to display results in real time. As per described above, the display module can include means to facilitate the connection of the module to the system such as at least one of a hooking mechanism (e.g., snap-fit hooking mechanism, magnetic hooking mechanism, and bolting mechanism), a button to release the hooking mechanism; and an auto alignment mechanism aiming to be easily stacked to the bottom side of the principal system. FIG. 18B is a view of the display module (180) that can be connected to the principal system of the multimodal and modular optical apparatus. The display module (180) includes a hooking mechanism (181) to ensure an adequate connection between the display module and the apparatus, and a release button (182) to disconnect the display module from the apparatus. The display module also includes a universal connector (183) that ensures communication with the principal system. The display module may also include an alignment mechanism (184) to ensure the right alignment of the module with the principal system. The display module includes a display screen (185) to give the user access to a user interface.

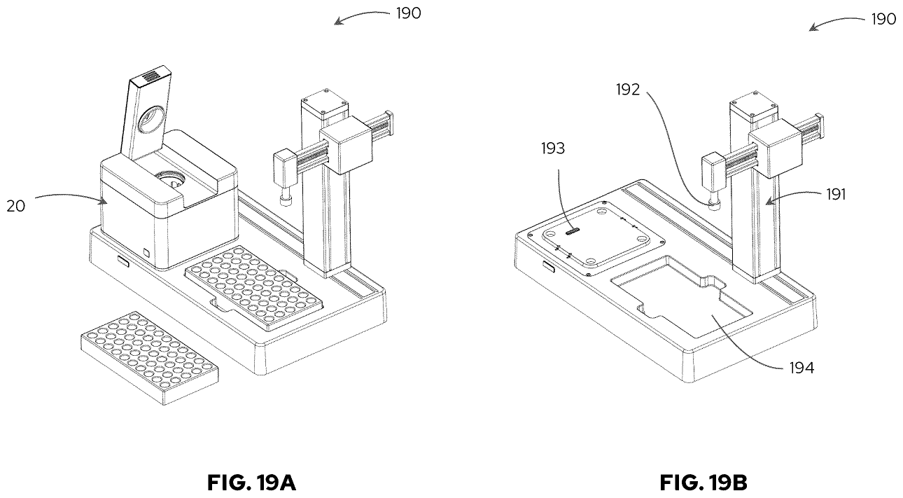

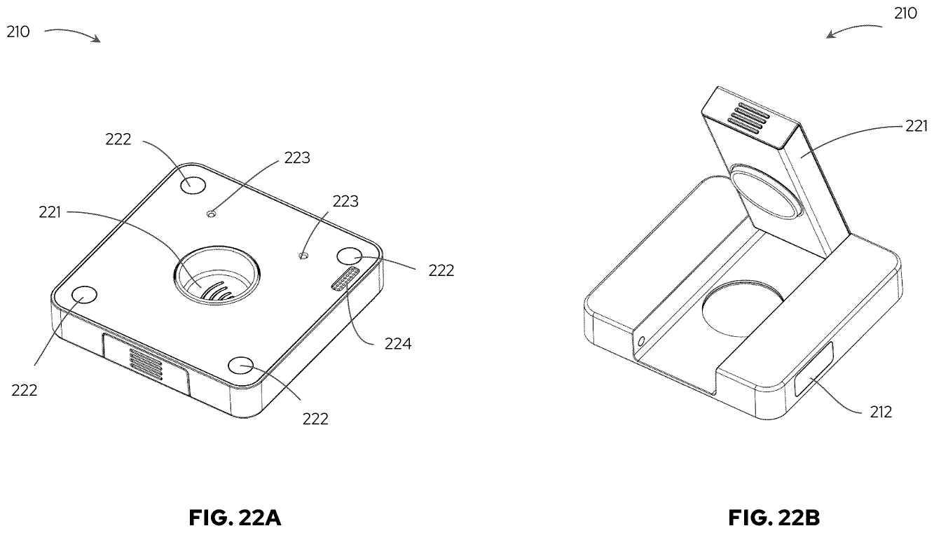

In some implementations, the multimodal and modular optical apparatus includes the automatic platform module being connected to the bottom universal connector of the principal system. The automatic platform module can be connected to the principal system of the multimodal and modular optical apparatus in order to handle material samples and automatically dispose them into the apparatus to perform the desired testing. For example, in addition to means for connecting a module to the principal system as described above, the automatic platform module can further comprise an arm robot or an automatic position system with at least three degrees of freedom and at least one gripper to handle sample containers aiming to automatically perform sample measurement using a multimodal and modular optical apparatus. FIG. 19A is a view of an automatic platform module (190) connected to the principal system (20) of the multimodal and modular optical apparatus (10). A top module (211, see FIGS. 22A and 22B) is further seen connected to the principal system (20). FIG. 19B is a view of the automatic platform module (190) alone. Referring to FIG. 19B, the automatic platform module (190) includes a multi-axis positioning system (191) that positions a gripper (192) intended to automatically handle and position material samples. The module (190) further includes a universal connector (193) that ensures the communication and optionally the power supply between the automatic platform module (190) and the principal system of the multimodal and modular optical apparatus. Optionally, the automatic platform module (190) can further comprise a temperature regulated bed (194) to regulate the temperature of the set of material samples that are handled by the automatic platform module (190) and tested by the principal system of the multimodal and modular optical apparatus.

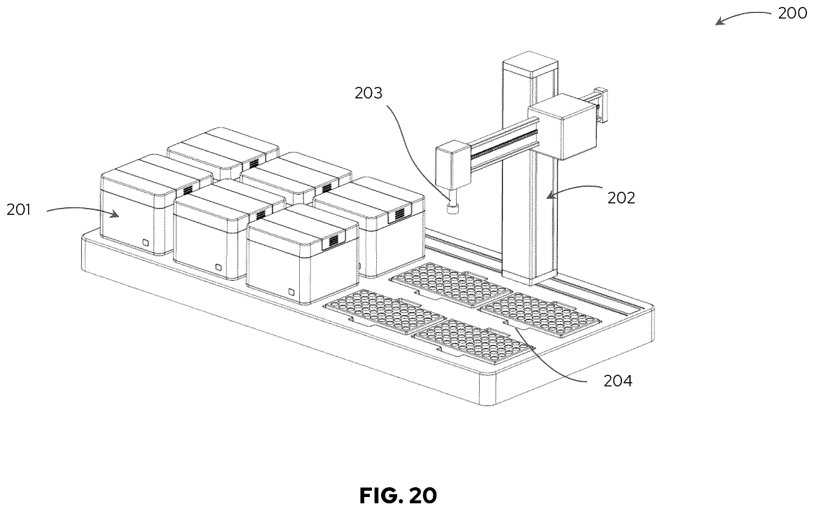

In some implementations, the multimodal and modular apparatus can be provided along with additional multiple multimodal and modular apparatuses to form an assembly, with a grid automatic platform module being connected to the principal system of each apparatus of the plurality of multimodal and modular optical apparatuses. The resulting assembly can handle material samples and automatically dispose them into the different apparatuses to perform the desired testing at a high-throughput. The grid automatic platform module comprises an arm robot or an automatic position system with at least three degrees of freedom and at least one gripper to handle sample containers for an automatic sample measurement using a multimodal and modular optical apparatus; at least one hosting area, preferably 6 hosting areas, to host a set of multimodal and modular optical apparatuses. The hosting area comprises a hooking mechanism (e.g., snap-fit hooking mechanism, magnetic hooking mechanism, and bolting mechanism); an auto alignment mechanism aiming to be easily stacked to the bottom side of the principal system. FIG. 20 illustrates a view of an automatic platform module (200) containing a set of principal systems of the multimodal and modular optical apparatus (201), a multi-axis positioning system (202) that positions a gripper (203) intended to automatically handle and position material samples. The grid automatic platform module may include a set of universal connectors that ensure the communication and potentially the power supply between the automatic platform module (200) and the set of principal systems of the multimodal and modular optical apparatus. The automatic platform module (200) may also contain a set of temperature regulated beds (204) to regulate the temperature of sets of material samples that are handled by the automatic platform module (200) and tested by the principal system of the multimodal and modular optical apparatuses.

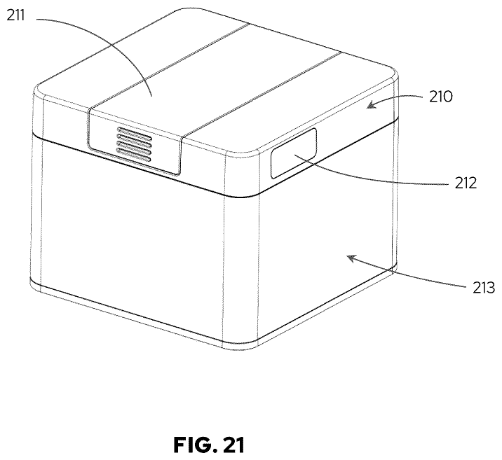

In some implementations, the multimodal and modular optical apparatus includes a multi-identification module being connected to a top surface of the principal system via a universal connector. As illustrated in FIG. 21, the multi-identification module (210) comprises a reader (211) and a reading window (212) for connection to the principal system (20) of the multimodal and modular optical apparatus (213).

More particularly, referring to FIG. 22A, the multi-identification module (210) can comprise at least one identification reader (211). For example, the at least one reader can comprise a Radio Frequency Identification (RFID) reader, a Near Field Communication (NFC) reader, a Quick Response (QR) code reader, a barcode reader, a proximity sensor, a motion sensor, or any combinations thereof. The multi-identification module (210) comprises a hooking mechanism (222) (e.g., snap-fit hooking mechanism, magnetic hooking mechanism, and bolting mechanism) and an alignment mechanism (223) aiming to be easily stacked onto the top side of the principal system. The multi-identification module (210) comprises a universal connector (224) to ensure the communication between the module and the principal system. As illustrated in FIG. 22B, the multi-identification module (210) can read, through the reading window (212), the ID of the sample to be tested in a multimodal and modular optical apparatus or actuate the opening of the reader (221) cover using a proximity or motion sensors. The multi-identification module (210) may comprise a motorized system to automatically open the cover and access the interior of the analysis chamber of the principal system.

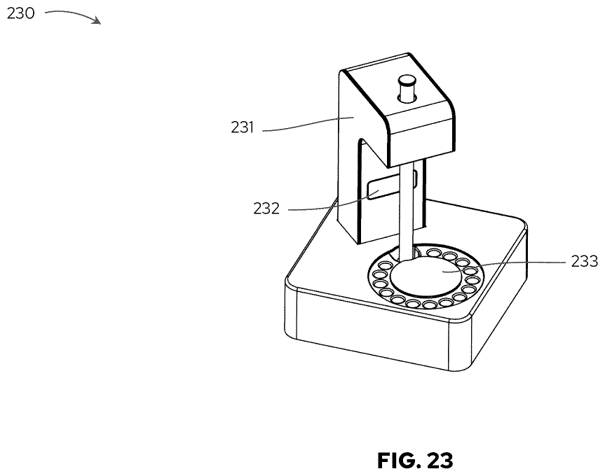

In some implementations, the multimodal and modular optical apparatus includes the carousel dispensing module being connected to a top surface of the principal system via a universal connector. The carousel dispensing module may be connected to the principal system of the multimodal and modular optical apparatus in order to automatically dispense material samples in the principal system. As illustrated in FIG. 23, the carousel dispensing module (230) may comprise a multi-axis positioning system (231), a reading window (232) and a rotating carousel (233).

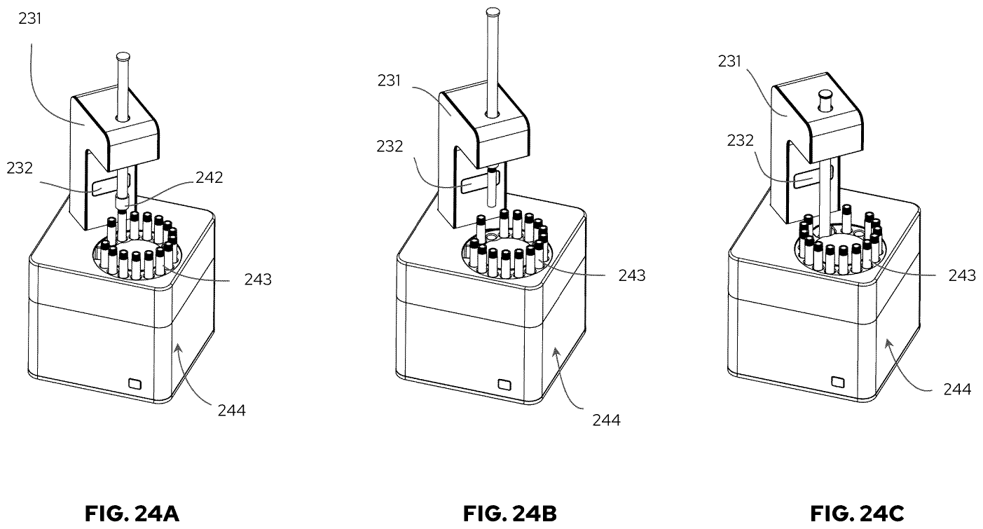

As illustrated in FIGS. 24A, 24B and 24C, the automatic carousel dispensing module comprises a rotary carousel for hosting sample containers and being used to position each sample container above the opening of the analysis chamber of the principal system. The automatic carousel dispensing module (230) comprises a multi-axis automatic position system (231), for example a vertical linear stage, ended by a rotating sample container gripper (242) to handle, rotate and move individually sample containers (243) from the carousel to the sample container adapter inside the analysis chamber (not shown) of the principal system of the multimodal and modular optical apparatus (244). The automatic carousel dispensing module (230) further comprises at least one reading window (232) containing an identification reader among the following: Radio Frequency Identification (RFID) reader, Near Field Communication (NFC) reader, Quick Response (QR) code reader or barcode reader. It should be noted that as per other modules, the automatic carousel dispensing module can further comprise a hooking mechanism (e.g., snap-fit hooking mechanism, magnetic hooking mechanism, and bolting mechanism), and optional auto alignment mechanism aiming to be easily stacked to the top side of the principal system. The automatic carousel dispensing module can be used to mix the sample contained into the sample container before installing the sample container in the analysis chamber of the apparatus.

In some implementations, one or more of the following module can be connected alone or in combination to the principal system of the multimodal and modular optical apparatus:
Imaging module: A module to image a sample such as a microscope. Optical arrangements can be used to image in real time the microscopic structure of a sample during measurements.
Liquid circulation module: A module comprising a pump to manage the circulation of a liquid in a tubing arrangement connected to a sample container for further analysis.
Gas injection module: A module comprising a pipe arrangement under pressure to inject gas and allow gas circulation inside a sample container for further analysis.
Drop analysis module: A module comprising optical arrangements to guide the light generated by the emitter cartridge to the drop sample (very low volume of sample) and to guide the resulting light to the receiver cartridge for further analysis.
Additional sensor module: A module comprising physico-chemical sensor readers (e.g., temperature, pH, etc.) that measured in real time physico-chemical parameters of the material sample using dedicated probes in contact or without contact with the sample.
Light stimulation module: A module comprising a light source and optical arrangements to generate light with wider power range, wider wavelength ranges, narrowed wavelength range, higher intensity, etc., that stimulate the sample material during or between measurements.

### Control and Processing Implementations

The controller and processing unit of the principal system of the multimodal and modular apparatus (or of each principal system of an assembly of multimodal and modular apparatuses) can be configured to communicate in real time with a plurality of cartridges, a plurality of modules (when provided) and analysis chambers (when provided). The controller and processing unit can be configured to communicate in real time with a user device using an Internet of Things (IoT) communication interface through a wired or wireless network. The controller and processing unit can be configured to process a raw optical signal (optical data) measured by at least one light detector and generate (via extraction, processing and/or calculation) optical or physical parameters that are correlated to the optical data. The generated parameters can include turbidity, nephelometric turbidity units, backscatter units, nephelometric turbidity multibeam units, monochrome light attenuation units, ratio white light turbidity units, optical density, absorbance, transmittance, fluorescence intensity, fluorescence spectra, absorption spectra, colorimetry, luminescence, fluorescence polarization, time-resolved fluorescence, particles size, or any combinations thereof.

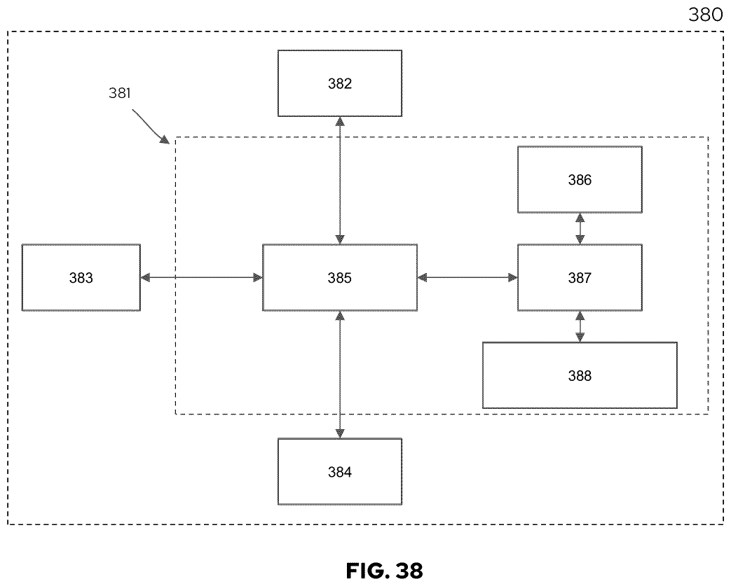

FIG. 38 illustrates the multimodal and modular optical apparatus (380). The principal system may include a control and processing unit (381) that may be configured to communicate, via a hardware manager (385), with analysis chamber (383) and/or cartridges (384) of the system and with external modules (382) (when provided). In some embodiments, the controller and processing unit (381) may also comprise a processor (387) and a memory (386) that may be in communication with or otherwise control a IoT communication interface (388). As such, the control and processing unit (381) may be embodied as a circuit chip (e.g., an integrated circuit chip) configured (e.g., with hardware, software or a combination of hardware and software) to perform hardware control, data processing, and network communication.

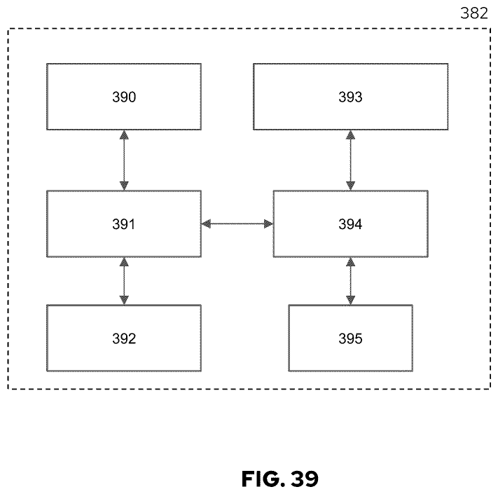

FIG. 39 illustrates the module (382) of FIG. 38 that may include actuators (390) to perform automatic operation such as handling, displaying, scanning, storing, etc., sensors (392) to perform a plurality of measurements such as temperature, images, light, humidity, gas, etc., connected to a hardware manager (391). In some embodiments, the module (382) may also comprise a secondary control and processing unit comprising a processor (394), a memory (395) and a communication interface (393) that may be in communication with the control and processing unit (381) of the principal system.

### Assembly/Network Implementations

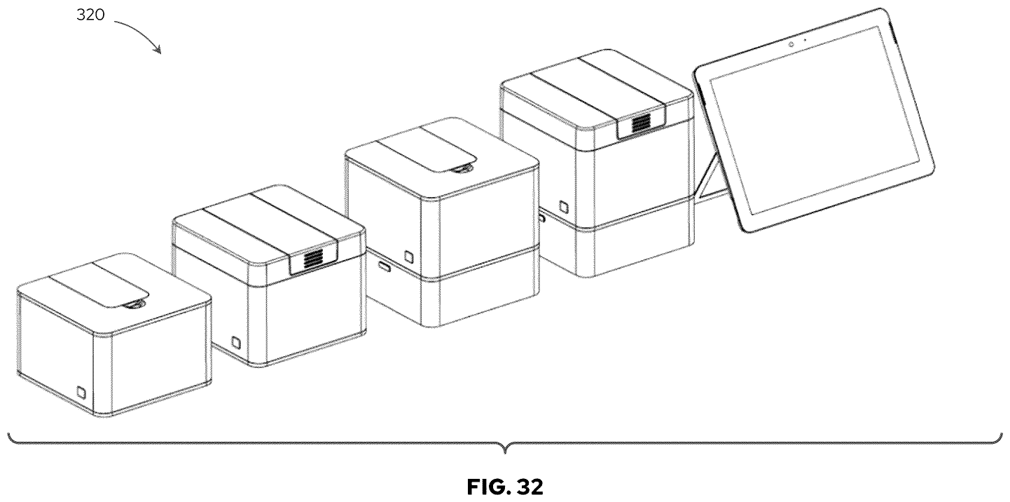

As shown in FIG. 32, there is further provided an assembly including multiple multimodal and modular optical apparatuses, each possibly having different optical configurations in terms of wavelengths and positions of the cartridges. One or more of the multimodal and modular optical apparatuses can have its principal system being individually connected to one or more additional modules as described herein. The apparatuses can be remotely interconnected, thought a wireless communication protocol such as WIFI or Bluetooth, to form a connected apparatus network (320) (assembly) that communicate in real time with a user device (i.e. desktop computer, laptop computer, server, tablet, cellphone, etc.) that incorporates a dedicated software and/or user interface.

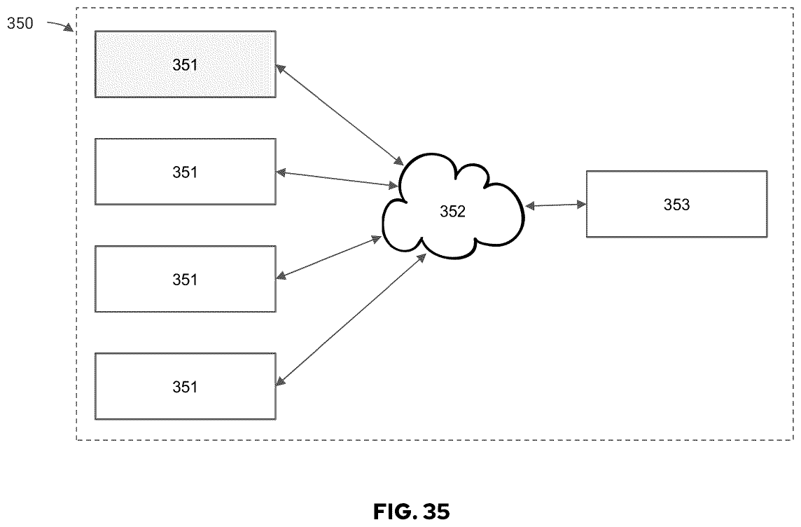

As illustrated in FIG. 35, a single multimodal and modular apparatus or an assembly of multimodal and modular apparatuses can be deployed in at least one geographic site/location (e.g., via a single site or multiple single sites) (351) and each geographic site/location. All apparatuses from the one or more geographic sites (351) can be connected to the internet to form a larger network of multimodal and modular optical apparatuses. The network of connected apparatuses (as illustrated for example in FIG. 32) can operate following a multisite system architecture (350) where the data (raw and/or generated as parameters) and metadata are stored in a common database (353) that is connected to the network based on this multisite system architecture via the internet (352).

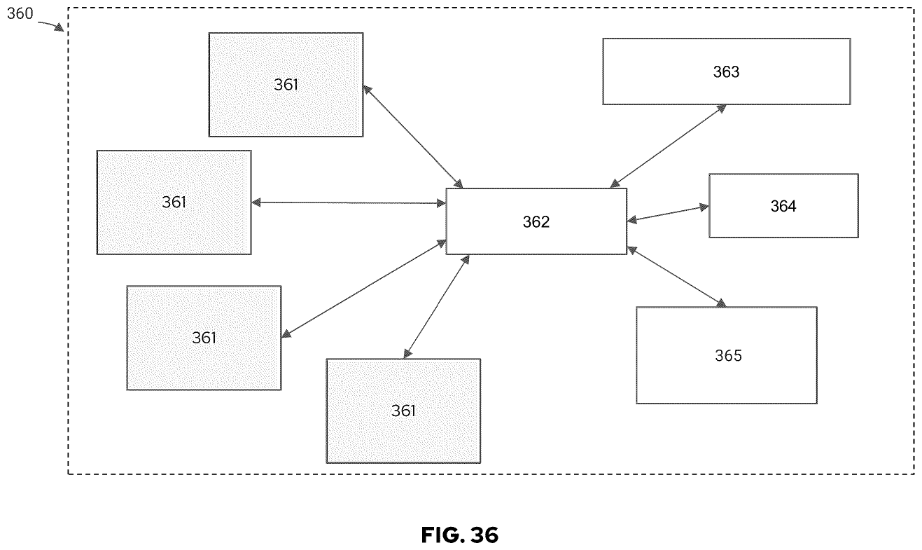

In an embodiment, as illustrated in FIG. 36, there is provided an assembly including one or more multimodal and modular optical apparatus(es) (361) operating based on a single site system architecture (360) that further comprises a user device (i.e., desktop computer, laptop computer, tablet, cellphone, etc.) (363) communicating in real time with the apparatus(es) (361) through a wired or wireless internal communication network (362) of the assembly. This assembly can further comprise a database (364) that aggregates the data (raw and/or generated as parameters) and metadata coming from the one or more multimodal and modular optical apparatus(es) (361). Using this network, each multimodal and modular optical apparatus can be connected to a robot or any other automatic platform (365) that can be further included in the assembly.

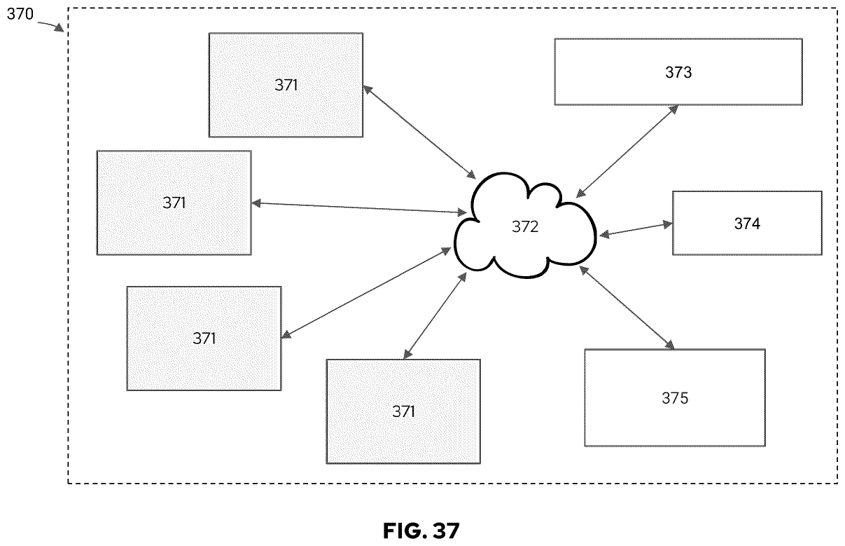

In another embodiment, as illustrated in FIG. 37, there is provided an assembly/network comprising one or more multimodal and modular optical apparatus(es) (371) operating based on another single site system architecture (370), and further comprising a user device (i.e., desktop computer, laptop computer, tablet, cellphone, etc.) (373) communicating in real time with the one or more apparatuses through Internet (372). This assembly/network further comprises a database (374) that aggregates the data (raw and/or generated as parameters) and the metadata coming from the one or more multimodal and modular optical apparatus(es) (371). Each multimodal and modular optical apparatus (371) can be connected to a robot or any other automatic platform (375) that is included in the assembly/network.

In another embodiment, there is provided an assembly/network of multimodal and modular optical apparatus(es) comprising one or multiple multimodal and modular optical apparatus(es) that communicate in real time with a user device (i.e., desktop computer, laptop computer, tablet, cellphone, etc.) through a wired or wireless internet. (FIG. 37)

Method for Measuring Data from the Material Sample and Generating at Least One Parameter Characterizing a Physical/Chemical/Biological Property of the Material

Considering the optical configuration (such as wavelength, emitting, receiving, spectroscopy, imaging, etc.) and the position of the cartridges around the sample container related to the emitted light path, the apparatus can be used as an absorbance meter, a transmittance meter, a colorimeter, a turbidimeter, a nephelometer, a spectrophotometer, a backscatter meter, a fluorometer, plate reader, etc. or any combination of these optical measurement modalities.

In some implementations, from the measurement performed by the cartridges at different angles and modalities, composite measurement parameters comprising one or more measurement(s) at different angles can be calculated by the control and processing unit to improve the sensitivity and the precision of the optical measurements. Considering the position of the cartridges at different angles with respect to the emitted light path, a more robust and more precise measurement can be obtained by switching automatically from one optical measurement modality to another optical measurement modality according to the optical characteristics of the sample to analyze. Optionally, in addition to light stimuli, other external stimuli such as temperature, gas, etc. can be simultaneously provided by dedicated cartridges and/or modules. The optical parameters of the material sample can thus be analyzed in presence of light, temperature, gas, etc. as a function of time, to study the sample evolution for a period of time, or at a specific time.

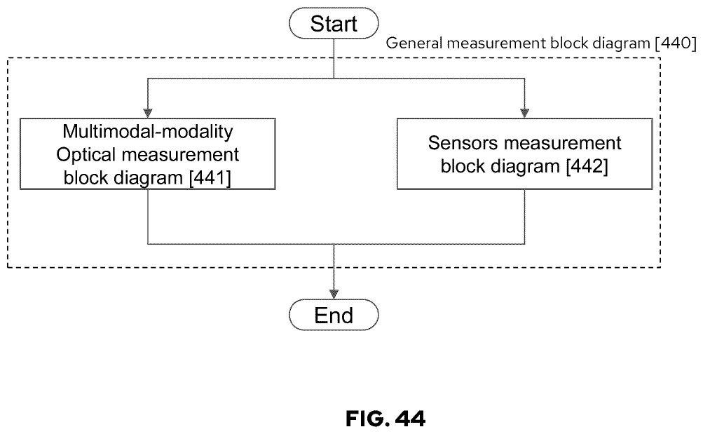

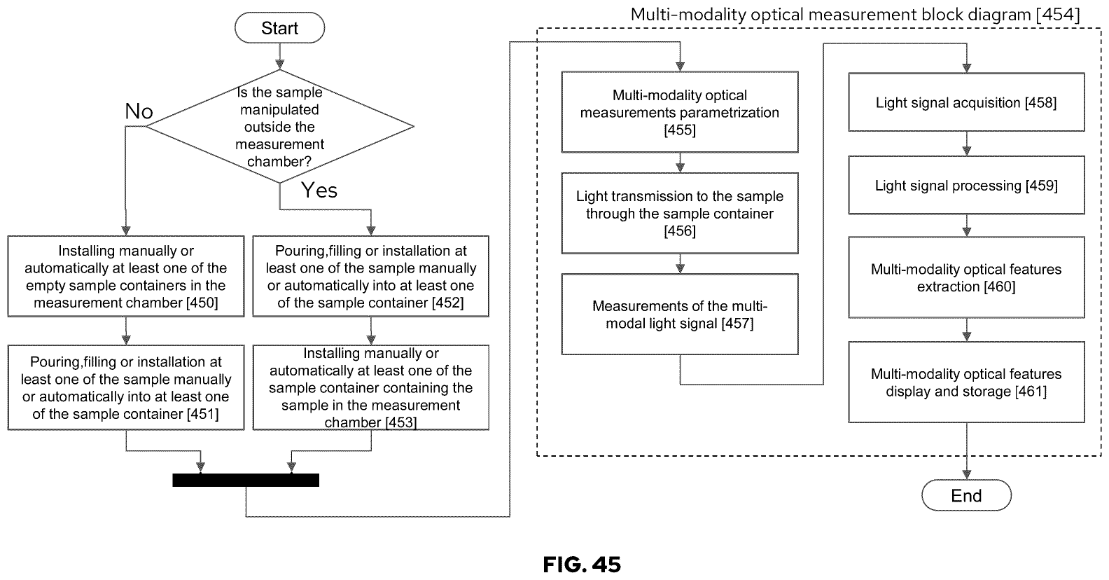

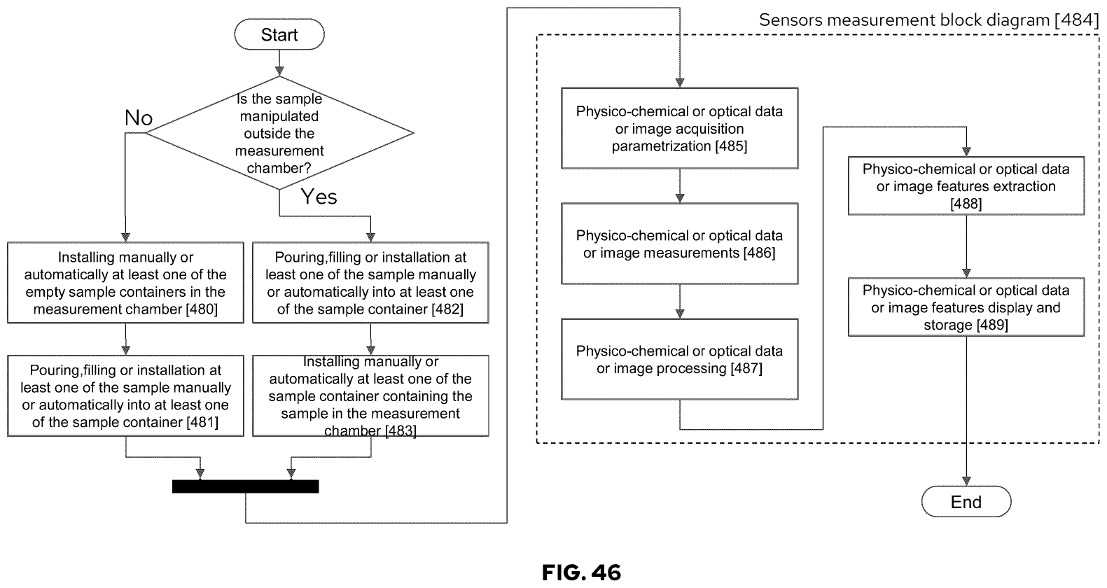

FIG. 44 presents a flowchart describing a general measurement block diagram (440) composed of the multi-modality optical measurement block diagram (see FIG. 45) and the sensor measurement block diagram (see FIG. 46). Accordingly, the system and methods of the present invention can enable the real-time combination of multi-modal measurements including optical data or image measurement to generate physico-chemical parameters, thereby increasing the amount of information that can be used to characterize a product or control its production.

FIG. 45 is a flowchart of a typical multi-modality optical measurement on a material sample using the apparatus of the invention. For a sample manipulated inside the measurement chamber, the first step (450) consists in installing an empty sample container in the measurement chamber. This operation can be done manually, semi-automatically or automatically. The second step (451) consists in pouring, filling or installing manually, semi-automatically or automatically the sample in the sample container.

For a sample manipulated outside the measurement chamber, the first step (452) consists in pouring, filling or installing manually, semi-automatically or automatically the sample in the sample container. Then, the second step (453) consists in installing manually, semi-automatically or automatically the sample container containing the sample in the measurement chamber. After multi-modality optical measurements parameterization (455) which is the configuration of the system to adjust parameters such as intensity, duration of excitation, modulation, etc., of the electromagnetic radiations that are emitted by the emitters of multiple cartridges. The electromagnetic radiations are transmitted (456) to the material sample through the sample container and sensors (receivers) measure (457) the electromagnetic response around the material sample (i.e., electromagnetic radiations from the material sample in response to stimulation by the emitted electromagnetic radiations from the emitters). The optical data corresponding to the electromagnetic response resulting from the combination of the selected optical measurement modalities (e.g., from the cartridges positioned at different angles) is acquired (458) and transmitted (459) to the control and processing unit for the signal conditioning. The data is then processed and multi-modality optical parameters are extracted (460) and serve to characterize the optical properties of the material sample. Finally, the results (and optionally related data) are either stored, shared or displayed or a combination thereof (461). The steps (455), (456), (457), (458), (459), (460) and (461) define the multi-modality optical measurement block diagram (454) which can be integrated in other functionalities of the multimodal and modular optical apparatus.

FIG. 46 presents a flowchart describing the determination of physical/chemical/biological parameters based on optical data and other sensor (physico-chemical) data such as temperature, pH, humidity, color, static/dynamic images, and the like of the sample. After dispensing (481) or (482) the material sample into the sample holder and the connection (480) or (483) of the sample container to the measurement chamber, the sensors measurement or block diagram (484), including the steps (485), (486), (487), (488) and (489), is activated. This block diagram describes an exemplary series of steps including parameterization of optical data (and optional physico-chemical data) acquisition (485), the actual measurement of the raw data (486) and the data post-processing (487) to condition the raw data into parameters. Several features of the parameter(s) are then extracted (488) such as the evolution over time, the activation time, phases identification and characterization, the maximum and minimum values, the increasing or decreasing rate, the image labelling or any other features describing the static or dynamic evolution (kinetics) of the measured parameter(s). Finally, the results are stored, shared and/or displayed (489).

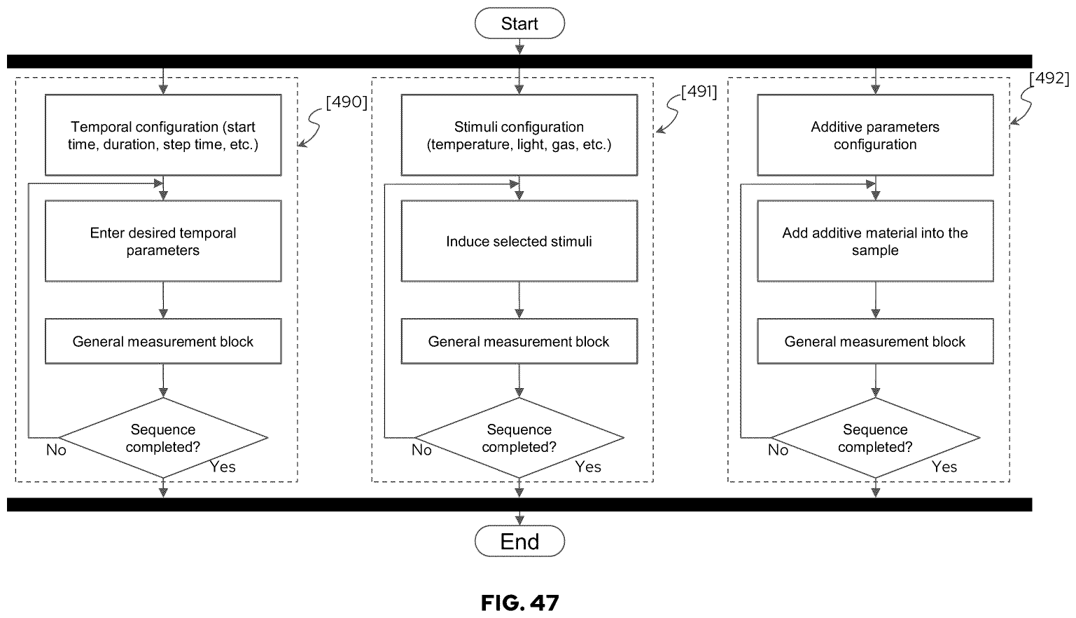

FIG. 47 is a more detailed flowchart that presents different configurations of data acquisition and treatment. The steps included into the step group (490) allows configuring the time parameters and allows configuring the delay between acquisitions, the number of acquisitions, the test duration or any other temporal conditions. The second configuration (491) is related to the stimuli configuration applied on the sample such as the temperature, the light, the gas, the humidity, or any other stimuli conditions. The third configuration (492) is related to the additive parameters that can be mixed or added to the material sample before, during and after the material characterization. For example, reagents, medication molecules, enzymes, coagulant agents, biochemical products, living cells or tissues, or any other substances of interest. All the above-mentioned configurations can be interlinked with each other (example: a change in temperature may trigger addition of a reagent) and are repeated until all the pre-defined stimuli conditions are sequentially satisfied. Several features are then extracted like the evolution over time of optical properties, the activation time, phases identification and characterization, the maximum and minimum values, the increasing or decreasing rate or any other features describing the measured kinetic. Finally, the results are stored, shared and displayed.

### Experimental Results

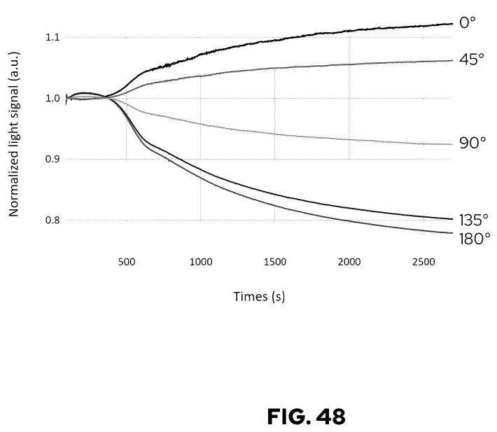

FIG. 48 is a graph of the normalized light signal as a function of time of a coagulating milk sample measured at different angles with respect to the light path. These measurements were performed using one emitter-receiver cartridge positioned at an angle of 0° (Backscatter) and four receiver cartridges positioned at angles of 45°, 90° (nephelometry), 135° and 180° (turbidimetry) with respect to the light path. The wavelength of the light generated by the emitter-receiver cartridge was 635 nm. A volume of 1 mL of 3.25% fat matter milk was renneted at 0.04% (diluted rennet in water) and poured in a sample container (glass vial) prior to start measurement. The sample container was installed in the vial sample container adapter and measurement points were acquired at a temperature of 32° C. during 2700 seconds every 2 seconds. The temperature of the sample was regulated using the thermo-optical analysis chamber which was connected to the thermal module. Each measurement angle provides information related to the gel formation. Measurements done at angles of 90°, 135° and 180° show lower normalized light signals that decrease at different rates over time whereas measurements at angles 0° and 45° exhibit an increase of the normalized light signal as a function of the milk coagulation.

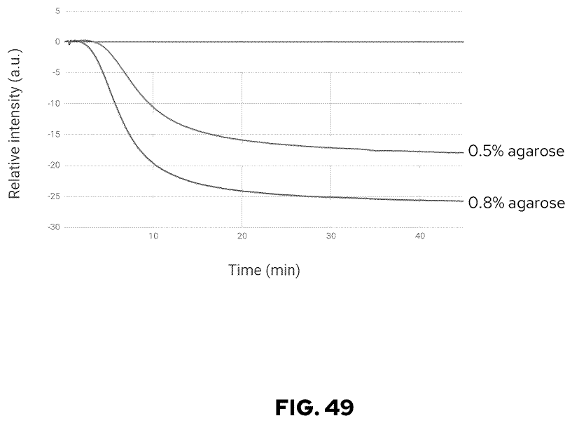

FIG. 49 is a graph of the relative intensity as a function of time of agarose gels formation. These measurements were performed at an angle of 180° (turbidimetry) with respect to the light path. The wavelength of the light was 880 nm. Agarose was diluted in water. Two different concentrations were tested (0.5% and 0.8%). A volume of 2 mL of sample was poured in a glass vial prior to measurement. The higher concentration sample exhibiting lower relative intensity at longer times.

It should be noted that for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional, and are given for exemplification purposes only. Therefore, the descriptions, examples, methods and materials presented in the claims and the specification are not to be construed as limiting but rather as illustrative only.

It is worth mentioning that throughout the following description when the article “a” is used to introduce an element it does not have the meaning of “only one” it rather means of “one or more”. For instance, the unit according to the invention can be provided with one or more reaction and/or separation chamber, one or more confining openwork structure, etc. without departing from the scope of the present invention. It is to be understood that where the specification states that a component, feature, structure, or characteristic “may”, “might”, “can” or “could” be included, that particular component, feature, structure, or characteristic is not required to be included.

Although the embodiments of the apparatus and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the apparatus, as will be briefly explained herein and as can be easily inferred herefrom by a person skilled in the art. Moreover, it will be appreciated that positional descriptions such as “above”, “below”, “top”, “bottom”, “upper”, “lower” and the like should, unless otherwise indicated, be taken in the context of the figures and should not be considered limiting.

In the following description, the term “about” means within an acceptable error range for the particular value as determined by one of ordinary skills in the art, which will depend in part on how the value is measured or determined, i.e., the limitations of the measurement system. It is commonly accepted that a 10% precision measure is acceptable and encompasses the term “about”.

In the above description, an embodiment is an example or implementation of the inventions. The various appearances of “one embodiment,” “an embodiment”, “some embodiments”, “certain embodiments”, or “some implementations” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

1. A multimodal and modular optical apparatus for acquiring optical data from a material sample and generating at least one parameter for characterization of the material sample, the apparatus comprising:
a principal system comprising:
a main body defining a sample receiving space and at least one cartridge-receiving space, wherein the sample receiving space is sized and shaped for receiving the material sample; and
at least one cartridge being sized and shaped for reversible insertion in the at least one cartridge-receiving space, wherein the at least one cartridge comprises:
an emitter configured to emit electromagnetic radiations propagating to the material sample, and/or a receiver configured to receive and measure electromagnetic radiations from the material sample in response to the emitter, thereby generating the optical data, and
a cartridge connector configured to communicate the optical data; and
a control and processing unit being in data communication with the at least one cartridge via the cartridge connector to receive the optical data, and further generating the at least one parameter for characterization of the material sample from the optical data.

2. The apparatus of claim 1, further comprising a sample container being sized and shaped to contain the material sample for insertion into the sample receiving space, wherein at least a portion of the sample container conducts/propagates the electromagnetic radiations.

3. The apparatus of claim 1, wherein the at least one cartridge comprises multiple cartridges being movable from one cartridge receiving space to another cartridge receiving space to propagate the electromagnetic radiations according to adjusted angles of emission/reflection.

4. The apparatus of claim 1, wherein the sample receiving space is one of:
central to the main body and the at least one cartridge receiving space comprises multiple cartridge receiving spaces being positioned peripherally around the sample receiving space;
an elongated channel and the principal system further comprises a conveyor at least partially encased in the sample receiving space, wherein the at least one cartridge receiving space comprises multiple cartridge receiving spaces being positioned on opposed longitudinal sides on the conveyor; and
an elongated channel and the principal system further comprises a positioning platform at least partially encased in the sample receiving space, wherein the at least one cartridge receiving space comprises multiple cartridge receiving spaces being positioned above or below the positioning platform.

5. The apparatus of claim 1, wherein the principal system further comprises an analysis chamber having an aperture to receive the material sample and being insertable in the sample receiving space.

6. The apparatus of claim 5, wherein the analysis chamber further comprises:
an actuator to expose the material sample to external stimuli; and/or
a sensor to measure a response of the material sample to external stimuli; and
a secondary control and processing unit, and a secondary connector to ensure at least one of data communication with and power supply to the control and processing unit via the secondary connector.

7. The apparatus of claim 1, wherein the main body further comprises an optical window being provided in alignment with at least one cartridge-receiving space to conduct/propagate the electromagnetic radiations to and from the at least one cartridge when inserted in the at least one cartridge-receiving space and through the main body.

8. The apparatus of claim 1, wherein at least one cartridge comprises at least one of:
an optical window to let a light beam go out of the cartridge or to let a light beam enter in the cartridge;
an additional sensor to measure physico-chemical data from the material sample; and
an emitter cartridge, a spot emitter cartridge, a linear emitter cartridge, an emitter-receiver cartridge, a receiver cartridge, a spot receiver cartridge, a linear receiver cartridge, a light stimulation cartridge, a Brownian motion cartridge, a Raman spectroscopy cartridge, a contactless temperature measurement cartridge, an imaging cartridge, or any combinations thereof.

9. The apparatus of claim 1, wherein the cartridge connector ensures electric power supply to the cartridge in addition to data communication.

10. The apparatus of claim 1, further comprising at least one module being operatively connected to the main body and being in data communication with the control and processing unit of the principal system, wherein the module comprises:
a module actuator to perform at least one automatic operation; and/or
a module sensor to perform measurement of additional physico-chemical data.

11. The apparatus of claim 10, wherein the at least one automatic operation comprises handling, displaying, sorting, scanning, regulating, controlling, acquiring data, storing data or any combinations thereof and the physico-chemical data comprises temperature, light, humidity, gas, image or any combinations thereof.

12. The apparatus of claim 10, wherein the module comprises at least one of:
mechanical components for hooking and/or alignment of the module with the principal system;
a secondary control and processing unit being connectable to the principal system via a universal connector for ensuring the data communication and the power supply; and
a thermal module, a battery module, a display module, an automatic platform module, a multi-identification module, a carousel dispensing module, an imaging module, a liquid circulation module, a gas injection module, a drop analysis module, a sensor module, a light stimulation module, or any combinations thereof.

13. The apparatus of claim 1, wherein the at least one parameter is at least one optical parameter comprising turbidity, nephelometric turbidity, optical density, absorbance, transmittance, fluorescence intensity, absorption spectra, or any combinations thereof.

14. The apparatus of claim 1, wherein the principal system is configured as an absorbance meter, a transmittance meter, a colorimeter, a turbidimeter, a nephelometer, a spectrophotometer, a backscatter meter, a fluorometer, an optical plate reader, an on-line optical apparatus, or any combinations thereof.

15. An assembly comprising multiple multimodal and modular optical apparatuses as defined in claim 1, each apparatus being configured for acquiring optical data from at least one material sample and generating at least one parameter for characterization of the at least one material sample; wherein the multiple apparatuses are in data communication with one another via their respective control and processing units.

16. A method to characterize a material sample based on optical data acquired by an apparatus having at least one optical measurement modality, the method including:
providing a material sample in a sample receiving space of the apparatus as defined in claim 1;
selecting the at least one optical measurement modality comprising inserting at least one cartridge in at least one cartridge receiving space of the principal system;
emitting and/or receiving the electromagnetic radiations via the at least one cartridge to generate the optical data from the material sample according to the selected optical measurement modality;
acquiring the optical data generated by the at least one cartridge in the control and processing unit of the principal system; and
generating the at least one parameter characterizing the material sample from the optical data in the control and processing unit.

17. The method of claim 16, comprising selecting another optical measurement modality to generate another parameter characterizing the material sample by performing at least one of:
releasing the at least one cartridge from the corresponding cartridge receiving space and inserting the at least one cartridge in another cartridge receiving space of the principal system;
inserting another cartridge in another cartridge receiving space of the principal system; and
releasing the at least one cartridge from the corresponding cartridge receiving space and inserting another cartridge in said cartridge receiving space of the principal system.

18. A process comprising monitoring at least one parameter characterizing a physical, chemical and/or biological property of a liquid material, wherein the monitoring comprises measuring optical data from the liquid material using the multimodal and modular optical apparatus as defined in claim 1, with the optical data being correlated to the at least one parameter.

19. The process of claim 18, wherein the multimodal and modular optical apparatus is as defined in claim 7; and the process further comprises communicating the monitored parameter to a control system and actuating at least one corrective action when the monitored parameter is off-specification.

20. The process of claim 18, wherein the at least one parameter is one of:
coagulation and the liquid material is milk;
microbiological fermentation or enzymatic coagulation, and the liquid material is animal milk or an alcoholic beverage during fermentation thereof; and
cell proliferation and the liquid material comprises at least one of cell cultures, microbes, or yeast.
